(19) United States
(12) Patent Application Publication
(10) Pub. No.: US 2011/0172625 A1
(43) Pub. Date: Jul. 14, 2011

(54) URINE RECEIVER AND WEARING ARTICLE

(75) Inventors: Ichiro Wada, Kagawa (JP); Miou Suzuki, Kagawa (JP); Rumi Yamaki, Kagawa (JP); Hiroko Murakami, Kagawa (JP)

(73) Assignee: UNI-CHARM CORPORATION, Ehime (JP)

(21) Appl. No.: 13/119,771

(22) PCT Filed: Sep. 17, 2009
(86) PCT No.: PCT/JP2009/066265

§ 371 Date: Mar. 18, 2011

(30) Foreign Application Priority Data
Sep. 19, 2008 (JP) 2008-241776
Sep. 19, 2008 (JP) 2008-241778
Sep. 19, 2008 (JP) 2008-241779

(51) Int. Cl. A61F 13/15

(52) U.S. Cl. 604/378; 604/384; 604/385.01

(57) ABSTRACT

The present invention provides a urine receiver being capable of quickly sucking urine discharged by the wearer.

A urine receiver includes a plurality of liquid-pervious sheets covering a top opening of a leakage-barrier receptacle and a pair of electrodes spaced from each other and parallelly extending in one direction P lies between each pair of the adjacent liquid-pervious sheets. Any one of the plurality of liquid-pervious sheets lying outside the pair of electrodes in a depth direction R of the receptacle is defined by a sheet 400 formed of a nonwoven fabric containing thermoplastic synthetic fibers. The sheet 400 is formed on its outer surface 400a with undulation comprising crests 401 and troughs 402 alternating in the one direction P and the crests 401 and troughs 402 extend in a direction intersecting with the one direction P across the pair of electrodes.

### TECHNICAL FIELD

A first aspect of the present invention relates to urine receivers adapted to be put on a person such as a bedridden aged one for whom it is difficult to control timing of urination and/or to make disposal of urine after discharged so that automatic urine disposal may be achieved.

Second and third aspects of the present invention relates to wearing articles and more particularly to wearing articles such as pants, disposable diapers or incontinent briefs provided with a suction unit for suction of moisture such as urine.

### RELATED ART

Conventionally, automatic urine disposal systems for a physically disabled person such as a bedridden aged one are known. For example, the urine receiver disclosed in JP 2006-26108 A (PATENT DOCUMENT 1) includes a suction unit to collect the urine discharged by the wearer and a urine guide tube extending from the suction unit to a vacuum tank. The suction unit has an opening covered with a liquid-pervious sheet and this liquid-pervious sheet is provided on its inner surface with a pair of electrodes spaced from and extending in parallel to each other. With the urine receiver put on the wearer's body, when the paired electrodes are electrically connected with each other by urine, electric signals are generated and, in response to this electric signal, a vacuum pump is actuated to cause urine evacuation from the suction unit toward the urine guide pipe to be initiated.

Conventionally, wearing articles such as pants provided on their side facing the wearer's skin with a moisture-suction system are known, for example, from JP 08-510924 A (PATENT DOCUMENT 2). In this PATENT DOCUMENT 2, a catheter system functioning to such and to evacuate urine outward from pants is described. In the case of this catheter system, the pants are provided on the side facing the wearer's body with an interface device comprising a plastic shell serving to receive urine, a liquid-impervious layer lying on the side of the shell facing the wearer's body and a hydrophobic cover stock material lying on the side of the liquid-impervious layer facing the wearer's body. A drain tube is connected to the shell so that urine staying in the pants may be collected into the shell under a suction effect and the urine collected into the shell may be evacuated from the pants through the drain tube.

Conventionally, wearing articles such as pants provided on their side facing the wearer's body with a urine sensor are known, for example, from JP 2004-41697 A (PATENT DOCUMENT 3). According to the disclosure of PATENT DOCUMENT 3, a urine sensor is sandwiched between a pair of diapers. The urine sensor includes a pair of electrodes extending in the longitudinal direction and, upon electrical connection of the paired electrodes via urine flowing between them, the urine sensor is turned on to detect occurrence of urination.

### PRIOR ART DOCUMENT

### Patent Document

PATENT DOCUMENT 1: JP 2006-26108 A
PATENT DOCUMENT 2: JP 08-510924 A
PATENT DOCUMENT 3: JP 2004-41697 A

### DISCLOSURE OF THE INVENTION

### Problem to be Solved by the Invention

In the urine receiver disclosed in PATENT DOCUMENT 1, a pair of electrodes is covered with the liquid-pervious sheet so that, upon occurrence of urination by the wearer of the urine receiver, urine may spread over this liquid-pervious sheet and/or the other liquid-pervious sheets and the paired electrodes may be electrically connected to each other via the liquid-pervious sheet(s) wetted with urine. However, it is not necessarily assured depending on the wearer's posture that the discharged urine smoothly spreads over the liquid-pervious sheet(s) and, in consequence, there is a possibility that the vacuum pump could not be actuated even when urination occurs.

It is an object of the present invention on a first aspect thereof to improve the known urine receiver so that a pair of electrodes may be electrically connected with each other via urine rapidly upon occurrence of urination.

The shell described in PATENT DOCUMENT 2 is made of a plastic material and therefore liquid-impervious. In addition, the liquid-impervious layer is formed on the side of the shell facing the wearer's body. Certainly, discharged urine is sucked away from the shell but a small quantity of urine is inevitably left in the shell without being sucked away. Vaporization of this small quantity of urine raises a temperature within the pants and makes the inside of the pants stuffy. Sweat of the wearer also may raise a temperature within the pants and cause stuffiness. Stuffiness generated within the pants may induce skin troubles such as eczema and create a feeling of discomfort against the wearer.

It is an object of the present invention on a second aspect thereof to assure the inside of the wearing article to be prevented from becoming stuffy and from causing skin troubles even when humidity within the wearing article rises.

The urine sensor disclosed in PATENT DOCUMENT 3 is designed to be held between a pair of diapers and the urine sensor may inevitably be displaced relatively to the diapers, for example, in the course of putting the diapers on the wearer's body or depending on the wearer's posture after the diapers have been put on the wearer's body. Urination can be detected at a high probability so far as the electrodes of the urine sensor are aligned with the urination region of the wearer but the urination detecting accuracy will be lowered if the urine sensor is displaced and, in consequence, the urine sensor will be out of alignment with the urination region of the wearer. However, even if the urine sensor is displaced relatively to the urination region of the wearer, it is impossible to check on the displacement because the urine sensor is held between a pair of diapers.

It is an object of the present invention on a third aspect thereof to provide the wearing article allowing the accuracy of moisture detection by a moisture detecting structure to be improved.

### Measure to Solve the Problem

According to the first aspect of the present invention, there is provided an urine receiver comprising a leakage-barrier receptacle having a bottom and a peripheral wall and a top opening, a urine evacuation duct extending through the peripheral wall adapted to be connected with urine suction means prepared separately of the receptacle, a plurality of liquid-pervious sheets stacked one on another to cover the top opening and a pair of electrodes spaced from each other and extending in one direction in parallel to each other wherein, in response to electrical connection of the pair of electrodes via urine contained in one or more of the plurality of liquid-pervious sheets, the urine suction means is actuated to suck urine discharged by the wearer into the receptacle through the top opening and to evacuate the urine sucked into the receptacle from the receptacle through the urine evacuation duct.

The first aspect of the present invention is characterized in that any one of the plurality of liquid-pervious sheets lying outside the pair of electrodes in a depth direction of the receptacle is formed of a nonwoven fabric containing thermoplastic synthetic fibers and has an outer surface facing outward in the depth direction and an inner surface facing inward in the depth direction wherein the outer surface is formed with undulation comprising crests and troughs alternating in the one direction and each of the crests and troughs extends across the pair of electrodes.

According to one embodiment of the first aspect of the present invention, the sheet formed on the outer surface thereof with the undulation has a flat inner surface and is put in close contact with the liquid-pervious sheet facing the flat inner surface.

According to another embodiment of the first aspect of the present invention, the sheet formed on the outer surface thereof with the undulation has the inner surface extending in parallel to the outer surface and is put in close contact with the liquid-pervious sheet facing the inner surface along respective bottoms of the troughs.

According to still another embodiment of the first aspect of the present invention, respective tops of the crests are hydrophobic and respective bottoms of the troughs are hydrophilic.

According to yet another embodiment of the first aspect of the present invention, the nonwoven fabric contains in the respective troughs thermoplastic synthetic fibers extending in the direction orthogonal to the one direction.

According to the second aspect of the present invention, there is provided an wearing article having a longitudinal direction and a transverse direction, comprising a side facing the wearer's body and a side facing the wearer's garment, a chassis having a front waist region, a rear waist region and a crotch region extending between the front and rear waist regions and a urine receiver adapted to receive bodily fluids discharged onto the chassis wherein the bodily fluids received by the urine receiver is sucked by a moisture sucking device out of the wearing article.

The second aspect of the present invention is characterized in that the receiver comprises a receptacle connected to the moisture suction device to collect the bodily fluids received by the receiver and a moisture-absorbent structure lying on the side of the receptacle facing the wearer's garment, and the moisture-absorbent structure comprises a moisture-pervious sheet lying on the side of the receptacle facing the wearer's body, a moisture-impervious sheet lying on the side facing the wearer's garment and a moisture-absorbent material sandwiched between the moisture-pervious sheet and the liquid-impervious sheet.

According to one embodiment of the second aspect of the present invention, the receiver additionally includes a moisture detecting structure comprising a moisture detecting sensor, a spacer lying on the side of the moisture detecting sensor facing the wearer's body, a dispersant sheet lying on the side of the moisture detecting sensor facing the wearer's garment and an air permeable-resistant sheet lying on the side of the dispersant sheet facing the wearer's garment.

According to another embodiment of the second aspect of the present invention, the moisture-absorbent structure has a length in the transverse direction dimensioned to be longer than that of the moisture detecting structure to extend outward in the transverse direction beyond the side edges of the moisture detecting structure.

According to still another embodiment of the second aspect of the present invention, the moisture detecting structure extends in the longitudinal direction from the front waist region to the rear waist region.

According to one embodiment of the second aspect of the present invention, the receptacle is liquid-impervious and flexible and may position in the rear waist region in use of the receptacle.

According to the third aspect of the present invention, there is provided a wearing article having a longitudinal direction and a transverse direction, comprising a side facing the wearer's body and a side facing the wearer's garment, a chassis having a front waist region, a rear waist region and a crotch region extending between the front and rear waist regions and a urine receiver adapted to receive bodily fluids discharged onto the chassis wherein the bodily fluids received by the urine receiver is sucked by a moisture sucking device out of the wearing article.

The third aspect of the present invention is characterized in that the receiver is formed with an alignment check mark adapted to be visually recognized from the outside through the chassis.

According to one embodiment of the third aspect of the present invention, the receiver has a longitudinal center line bisecting a length dimension in the transverse direction and the alignment check mark linearly extends in the longitudinal direction along the longitudinal center line.

According to another embodiment of the third aspect of the present invention, the alignment check mark comprises a first segment linearly extending along the longitudinal center line and a second segment having a length dimension in the transverse direction larger than that of the first segment.

According to still another embodiment of the third aspect of the present invention, at least in the crotch region, the receiver is visually recognized through the chassis. At least in the crotch region, the chassis is formed with windows through which the receiver is exposed.

According to yet another embodiment of the third aspect of the present invention, at least in the crotch region, the chassis is formed with windows through which the receiver is exposed.

According to further another embodiment of the third aspect of the present invention, the moisture detecting structure is formed so as to extend along the longitudinal center line of the receiver.

### Effect of the Invention

In the urine receiver according to the first aspect of the present invention, any one of the liquid-pervious sheets covering a pair of electrodes is formed of the nonwoven fabric of which the outer surface has the undulation comprising the crests and the troughs alternating in the direction in which the electrodes extend. These crests and troughs respectively extend in the direction intersecting with the direction in which the electrodes extend across the electrodes. Urine discharged by the wearer of the urine receiver flows into the troughs and flows along the troughs. In consequence, the electrodes can be quickly bridged by urine and electrically turned on. As a result, the urine suction means connected to the urine receiver also can be quickly actuated and suction of urine into the receptacle as well as evacuation of urine from the receptacle can be promoted.

According to the second aspect of the present invention, the receiver includes the moisture-absorbent structure serving to absorb moisture vapor generated within the wearing article. With such an arrangement, even if moisture vapor within the wearing article increases, such moisture vapor can be absorbed by the moisture-absorbent structure and thereby it is possible to restrict a rise of the temperature within the wearing article from rising. It is possible thereby to prevent the interior of the wearing article from becoming stuffy as well as to reduce a feeling of discomfort against the wearer.

According to the third aspect of the present invention, the alignment check mark adapted to be visually recognized is formed on the side of the receiver including the moisture detecting structure facing the wearer's body. Such an arrangement makes it possible to determine whether the receiver lies on a desired position or not. If the receiver is determined to be displaced from the desired position, the receiver can be repositioned so that the moisture detecting structure may be aligned with the desired position and thereby the desired detection accuracy of the moisture detecting structure may be assured.

### BRIEF DESCRIPTION OF THE DRAWINGS

FIG. 1 is a schematic diagram illustrating a construction of an automatic urine disposal system including a urine receiver.

FIG. 2 is a diagram showing the urine receiver as put on a wearer.

FIG. 3 is a plan view of the urine receiver.

FIG. 4 is a sectional view taken along line IV-IV in FIG. 3.

FIG. 5 is a sectional view taken along line V-V in FIG. 3.

FIG. 6 is a plan view of a urine sensor.

FIG. 7 is a sectional view taken along line VII-VII in FIG. 6.

FIG. 8 is a plan view of the urine sensor with insulating coating peeled off.

FIG. 9 is a perspective view of a strip of sheet.

FIG. 10 is a sectional view taken along line X-X in FIG. 9.

FIG. 11 is a view similar to FIG. 10, exemplarily showing a skin-contact sheet.

FIG. 12 is an overall diagram schematically illustrating the system according to the present invention.

FIG. 13 is a plan view of the urine receiver as viewed from its side facing the wearer's body.

FIG. 14 is a sectional view taken along line XIV-XIV in FIG. 13.

FIG. 15 is a sectional view taken along line XV-XV in FIG. 13.

FIG. 16 is a plan view of the urine receiver as viewed from its side facing the wearer's garment.

FIG. 17 is a perspective view of a urine receptacle.

FIG. 18 is a sectional view taken along line XVIII-XVIII in FIG. 17.

FIG. 19 is an overall diagram schematically illustrating the system according to a first embodiment of the present invention.

FIG. 20 is a plan view of the urine receiver as viewed from its side facing the wearer's body.

FIG. 21 is a sectional view taken along line XXI-XXI in FIG. 20.

FIG. 22 is a sectional view taken along line XXII-XXII in FIG. 20.

FIG. 23 is a plan view of the urine receiver as viewed from its side facing the wearer's garment.

FIG. 24 is a perspective view of the urine receptacle.

FIG. 25 is a diagram similar to FIG. 19, schematically illustrating the system according to a second embodiment of the present invention.

### IDENTIFICATION OF REFERENCE NUMERALS USED IN THE DRAWINGS

100a urine suction means
102 urine receiver
112 urine receptacle (container)
112a opening
112b bottom
112c peripheral wall
114 opening
143 electrode
143 electrode
134 liquid-pervious sheet (skin-contact sheet)
126 liquid-pervious sheet (dispersion sheet)
400 strip of sheet
400a outer surface (upper surface)
400b inner surface (lower surface)
401 crests
402 troughs
407 thermoplastic synthetic fibers
P longitudinal direction
Q transverse direction
R depth (thickness) direction
501 wearing article
502 chassis
503 receiver
504 moisture sensor structure
505 urine receptacle
506 moisture-absorbent structure
521 front waist region
522 rear waist region
523 crotch region
541 moisture sensors
542 spacer
549 dispersant sheet
550 air permeable-resistant sheet
562 moisture pervious sheet
563 liquid-impervious sheet
500P suction pump (moisture suction device)
601 wearing article
602 chassis
603 receiver
604 moisture sensor structure
605 urine receptacle (container)
606 mark
621 front waist region
622 rear waist region
623 crotch region
626 window
641 moisture sensor
661 first segment
662 second segment
600P suction pump

### DETAILED DESCRIPTION OF THE PREFERRED EMBODIMENTS

Details of a urine receiver according to a first aspect of the present invention will be more fully understood from the description given hereunder with reference to the accompanying drawings.

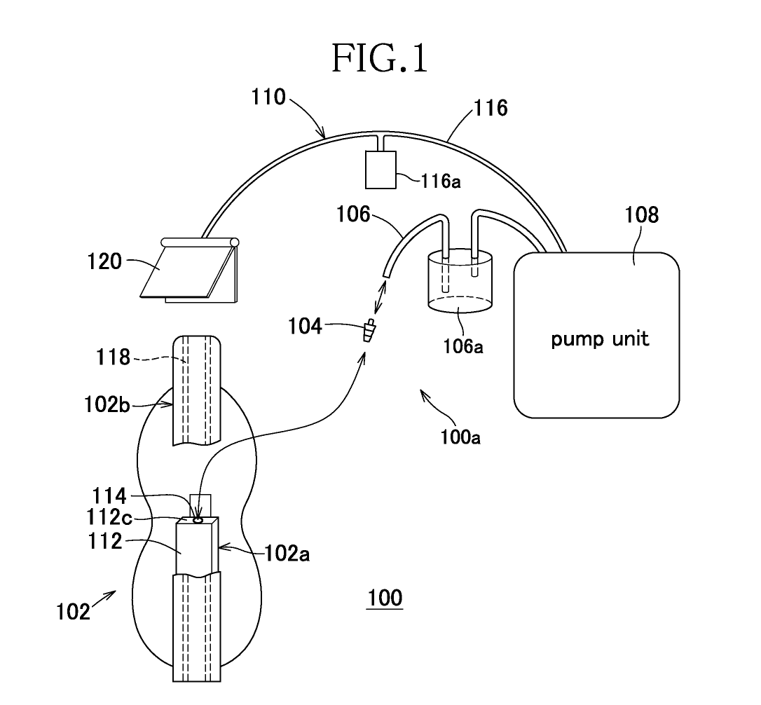

FIG. 1 is a schematic diagram illustrating an automatic urine disposal system 100 comprising a urine receiver 102 according to the present invention and vacuum-utilizing urine suction means 100a. The urine receiver 102 has a side facing the wearer's skin and a side facing the wearer's garment. In FIG. 1, various elements on the side facing the wearer's garment are illustrated as partially cutaway.

The automatic urine disposal system 100 can automatically collect urine discharged by the wearer into the urine receiver 102 upon urination. The urine receiver 102 comprises a urine collector unit 102a put in contact with the wearer's skin in the vicinity of the wearer's urethral orifice and adapted to collect discharged urine and a urine sensor unit 102b adapted to detect urination. The vacuum-utilizing urine suction means 100a includes a joint element 104 by which the suction means 100a is connected to the urine receiver 102, a urine guide tube 106, a urine reservoir 106a, a pump unit 108 and an electric wiring 116.

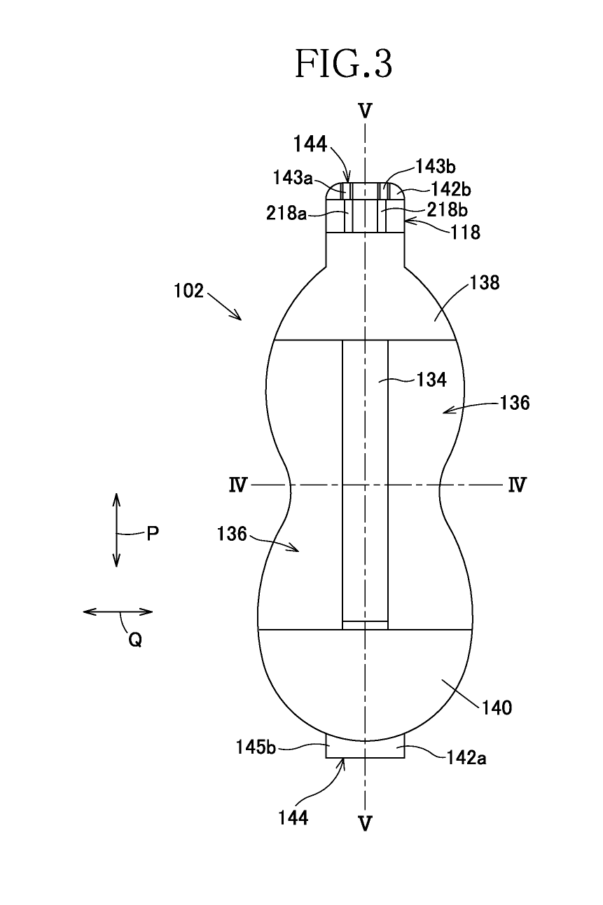

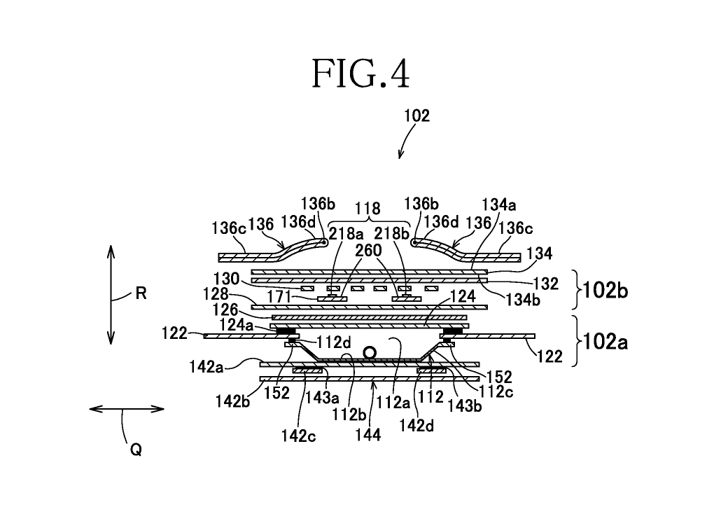

The pump unit 108 includes a suction pump (not shown) adapted to be actuated in response to signals transmitted via the wiring 116 from the urine sensor unit 102b. In the urine receiver 102, the urine guide tube 106 is connected via the joint element 104 to a urine evacuation port 114 formed through a peripheral wall 112c of a receptacle 112 in the urine collector unit 102a. A distal end of the wiring 116 extending from the pump unit 108 is provided with a clip 120 by which a pair of electrodes 218a, 218b (See FIGS. 3 and 4) of a urine sensor 118 included by the urine sensor unit 102b are electrically connected to the wiring 116. In such automatic urine disposal system 100, the urine sensor 118 detects occurrence of urination and generates signals on the basis thereof. In response to the signals transmitted from the urine sensor 118, the suction pump included by the pump unit 108 is actuated to suck air within the urine reservoir 106a and thereby to suck urine into the urine receptacle 112 so that the urine sucked in this manner may be further sucked and collected through the joint element 104 and the urine guide tube 106 into the urine reservoir 106a.

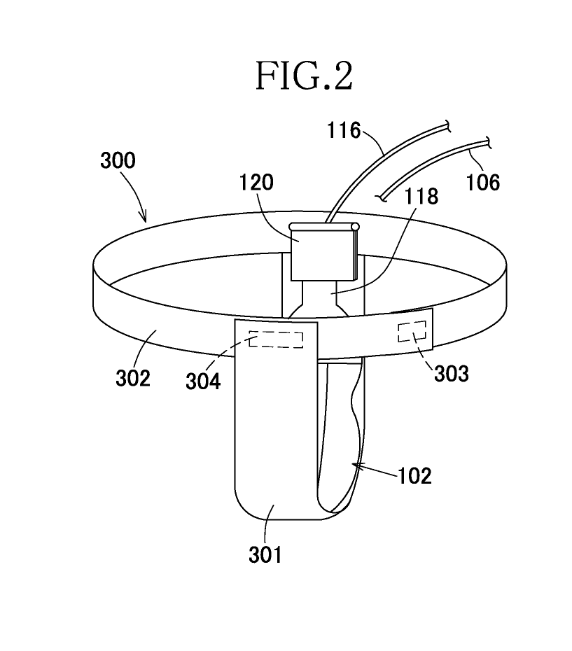

FIG. 2 exemplarily illustrates how to wear the urine receiver 102. With the urine receiver 102 put on the wearer's body, the clip 120 lies on the wearer's ventral side. A T-belt 300 is used as means to wear the urine receiver 102 on the wearer's body, the urine receiver 102 is fixed to the inner surface of a crotch belt segment 301 as a component of the T-belt 300 with pressure-sensitive adhesives or mechanical fasteners well known by the trade mark “Velcro” (registered trademark). Most part of the receptacle 112 in the urine receiver 102 extends in the vertical direction along the front side of the wearer's body so that the inner side of the urine receiver 102 may face the urethral orifice, the vicinity thereof and further extends along the inner surface of the crotch-belt segment 301 toward the anus in a gentle curve. Opposite ends of a waist-belt segment 302 as another component of the T-belt 300 are temporarily connected to each other by suitable connector means 303 such as mechanical fasteners. The crotch-belt segment 301 is stitched at one end together with the waist-belt segment 302 and temporarily connected at the other end to the waist-belt segment 302 with mechanical fasteners 304. It should be noted that the means to wear the urine receiver 102 is not limited to the T-belt 302 as illustrated, but the other appropriate means such as open-type diapers, pants-type diapers, diaper chassises or pants for incontinent patient also may be used for the same purpose.

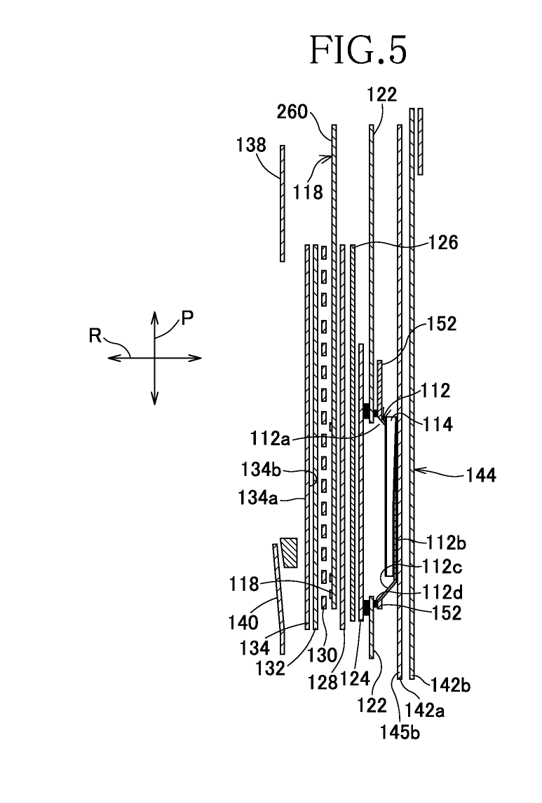

FIG. 3 is a plan view showing the side of the urine receiver 102 facing the wearer's skin (not shown), FIG. 4 is a sectional view taken along line IV-IV in FIG. 3 and FIG. 5 is a sectional view taken along line V-V in FIG. 3. In FIGS. 4 and 5, most of respective elements of the urine receiver 102 overlapping one another in the thickness direction R are shown to be spaced one from another for convenience of explanation. The thickness direction R may be reworded by the depth direction of the receptacle 112. As used herein, the term “outward in the depth direction” means the direction from the opening 112a of the receptacle 112 to the outside of the receptacle 112 and as used herein, the term “inward in the depth direction” means the direction from the opening 112a to the inside of the receptacle 112 as viewed in FIG. 4.

The urine receiver 102 has a longitudinal direction P corresponding to the vertical direction of the wearer's body and a transverse direction Q which is orthogonal to the longitudinal direction P. The urine receiver 102 has a width which is relative large in the vicinity of its opposite end sections as viewed in the longitudinal direction P and gradually reduced in its middle section. The urine receiver 102 has a thickness direction R also and includes, above the receptacle 112 as viewed in FIG. 4, a plurality of sheet-like elements, i.e., a liquid-impervious leakage-barrier sheet 122, an air permeable-resistant and liquid-pervious sheet 124, a dispersant sheet 126, a cushion sheet 128, the urine sensor 118, a sheet-like spacer 130, a sheet-like filter 132, a liquid-pervious skin-contact sheet 134 and leakage-barrier 136 stacked in this order from below. The leakage-barrier sheet 122, the air permeable-resistant sheet 124 and the dispersant sheet 126 are integrated with the receptacle 112 to form the urine collector unit 102a. The cushion sheet 128, the urine sensor 118, the spacer 130, the filter 132 and the skin-contact sheet 134 are stacked one on another to form the urine sensor unit 102b.

The receptacle 112 is leak-proof tray-shaped means contoured by a bottom 112b, the peripheral wall 112c and an upper opening 112a and made of soft elastic material such as soft polyethylene or silicon rubber. The receptacle 112 has flexibility of a degree such that the receptacle 112 can be inflected in the longitudinal direction P as well as in the transverse direction Q but resist against a deformation due to a negative pressure generated when the suction pump sucks urine. The leakage-barrier sheet 122 is water-tightly bonded to a peripheral flange 152 of the receptacle 112 with adhesive 112d.

The leakage-barrier sheet 122 extends outward beyond the peripheral edge thereof to prevent urine from leaking out of the urine receiver 102. The leakage-barrier sheet 122 may be formed of a thermoplastic synthetic resin film or a composite sheet consisting of such film and a nonwoven fabric. In the case of the urine receiver shown in FIG. 1, a soft polyethylene film having a thickness of 30 μm may be used for the leakage-barrier sheet 122. In cases where the urine receiver 102 requires no particular leakage-barrier effect along the periphery of the receptacle 112, the leakage-barrier sheet 22 may be spared from the elements required to exploit the present invention.

The air permeable-resistant sheet 124 is highly liquid-pervious but absolutely or substantially air-impermeable and covers the upper opening 112a of the receptacle 112 as seen in FIG. 4. The periphery of the sheet 124 is bonded to the leakage-barrier sheet 122 from above with hot melt adhesives 124a. A negative pressure can be easily generated within the receptacle 112 covered with the air permeable-resistant sheet 124 sufficiently to suck urine discharged onto the skin-contact sheet 134 rapidly upon actuation of the suction pump of the pump unit 108. The air permeable-resistant sheet 124 may be formed, for example, of an SMS nonwoven fabric consisting of a spun bonded nonwoven fabric having a basis mass of 22 g/m², a melt blown nonwoven fabric having a basis mass of 10 g/m² and a spun bonded nonwoven fabric having a basis mass of 22 g/m² preferably modified by surfactant so as to become hydrophilic. The air-permeability of the air permeable-resistant sheet 124 determined by Air-Permeability Measuring Method A specified by JIS L 1096, Section 6.27.1 is in a range of 0 to 100 cc/cm²/sec, more preferably in a range of 0 to 50 cc/cm²/sec in a wet condition and in a range of 20 to 100 cc/cm²/sec, more preferably in a range of 20 to 50 cc/cm²/sec in a dry condition. The wet condition in which the air-permeability is measured refers to a condition in which the moisture content of the air permeable-resistant sheet 124 calculated according to an equation as indicated below is 100% or higher. The dry condition means the condition of the air permeable-resistant sheet 124 after this sheet 124 has been left for 24 hours or longer in a room at a temperature of 20° C. and RH of 50%.

Moisture content (%)=(mass of sheet in wet condition−mass of sheet in a dry condition)/(mass of sheet in a dry condition) Equation (1)

The dispersant sheet 126 is formed of a liquid-pervious sheet such as a nonwoven fabric containing hydrophilic fibers, for example, rayon fibers and serves to disperse urine over the air permeable-resistant sheet 124 as rapidly as possible so that the air permeable-resistant sheet 124 may be wet over an area as wide as possible. Once the air permeable-resistant sheet 124 has been wetted, it is easy to generate a negative pressure within the receptacle 112 and thereby suck urine into the receptacle 112. Preferably, the dispersant sheet 126 is intermittently bonded to the air permeable-resistant sheet 124 to prevent liquid-pervious properties of these both sheets from being disturbed.

The cushion sheet 128 is formed, for example, of a liquid-pervious sheet such as a thermally bonded nonwoven fabric having a basis mass in a range of 20 to 30 g/m² and capable of picking up urine instantaneously at the initial stage of urination to prevent urine staying in the dispersant sheet 126 and the air permeable-resistant sheet 124 from flowing back toward the urine sensor 118. The sheet-like material such as the urine sensor 118, the spacer 130 and the filter 132 may be previously stacked on the cushion sheet 128 to utilize the cushion sheet 128 as a carrier element serving for previously arranging these sheet-like elements at predetermined positions in the urine receiver 102 in the course of producing the urine receiver 102. Preferably, the cushion sheet 128 is intermittently bonded to the dispersant sheet 126 to prevent the liquid-pervious properties of these both sheets from being disturbed.

The urine sensor 118 is obtained, for example, by printing a pair of electrodes each having a desired shape on a synthetic resin film with conductive ink and a detailed construction thereof will be described later. The urine sensor 118 may be located at least in a space defined between the air permeable-resistant sheet 124 and the skin-contact sheet 134 of a plurality of liquid-pervious sheets 124, 126, 128, 130, 132 and 134 and, in the illustrated embodiment, the urine sensor 118 is sandwiched by the cushion sheet 128 and the spacer 130. The urine sensor 118 may be bonded to the cushion sheet 128.

The spacer 130 is formed of a liquid-pervious mesh sheet. In the urine receiver 102, a certain quantity of urine should be left in the skin-contact sheet 134 and keep the skin-contact sheet 134 in a wet condition even after suction of urine. If such skin-contact sheet 134 still in a wet condition comes in direct or indirect contact with the urine sensor 118 under a body mass or the other factors, there is a possibility that the urine sensor might malfunction. The spacer 130 is means serving to keep the urine sensor 118 and the filter 132 to be spaced from each other in the thickness direction R and thereby to prevent such malfunction of the urine sensor 118. The spacer 130 is not urine-absorbent but water-repellent and has air-permeability, liquid-permeability and stiffness higher than those of the air permeable-resistant sheet 124 so that the spacer 130 may keep its thickness unchanged even under the body mass. Such spacer 130 may be formed of a mesh sheet having a thickness in a range of 0.5 to 1 mm made of a soft synthetic resin such as ethylene-vinyl acetate and bonded to the cushion sheet 128 preferably in such a manner that the liquid-permeably properties of these both sheets 130, 128 is not disturbed.

The filter 132 serves to avoid a situation in which solid contents of urine might cling to the urine sensor 118 and, in consequence, the urine sensor 118 might be permanently left in a turn-on state. The filter 132 may be formed of a sheet material, more preferably of a nonwoven fabric having air-permeability and liquid-permeability higher than those of the air permeable-resistant sheet 124. The filter 132 may be intermittently bonded to the spacer 130 to prevent the liquid-permeably properties of these both sheets 132, 130 from being disturbed.

The skin-contact sheet 134 has an upper surface 134a and a lower surface 134b wherein the lower surface 134b is kept in tight contact with the upper surface of the filter 132. With the urine receiver 102 put on the wearer's body, the upper surface 134a faces the wearer's urination orifice and the vicinity thereof and, in this state, is kept in contact with the wearer's skin. This skin-contact sheet 134 is formed of a sheet material having flexibility and liquid-permeability such as a thermally bonded nonwoven fabric having a basis mass in a range of 15 to 25 g/m². Like the cushion sheet 128, the skin-contact sheet 134 is capable of picking up urine instantaneously at the initial stage of urination and it is preferred to bond the skin-contact sheet 134 intermittently to the filter 132 so that the liquid-permeable property of these both sheets 134, 132 may be assured. The skin-contact sheet 134 may be hydrophilic or water-repellent depending on conditions.

Paired right and left leakage-barriers 136 seen in FIGS. 3 and 4 are capable of preventing urine from flowing on the skin-contact sheet 134 in the transverse direction Q and leaking sideways out of the urine receiver 102. Of the leakage-barriers 136, outer side edges 136c lying outside the urine receiver 102 are bonded to the skin-contact sheet 134 and inner side edges 136d lying in the vicinity of middle section of the urine receiver 102 are not bonded to the skin-contact sheet 134 wherein the inner side edges 136d are provided with elastic elements 136b such as rubber yarns (See FIG. 4) are attached thereto under tension in the longitudinal direction P. With the urine receiver 102 put on the wearer's body, the elastic elements 136b are curved in the longitudinal direction P as seen in FIG. 1 and thereupon and the inner side edges 136d of the leakage-barriers 135 are spaced upward from the skin-contact sheet 134. The leakage-barriers 136 are formed of a soft thermoplastic synthetic resin film or a composite sheet consisting of this film and a nonwoven fabric and are preferably liquid-impervious. As viewed in the plan view of the urine receiver 102, the leakage-barriers 136 (See FIG. 3) have upper and lower ends covered with first and second end sheets 138, 140, respectively.

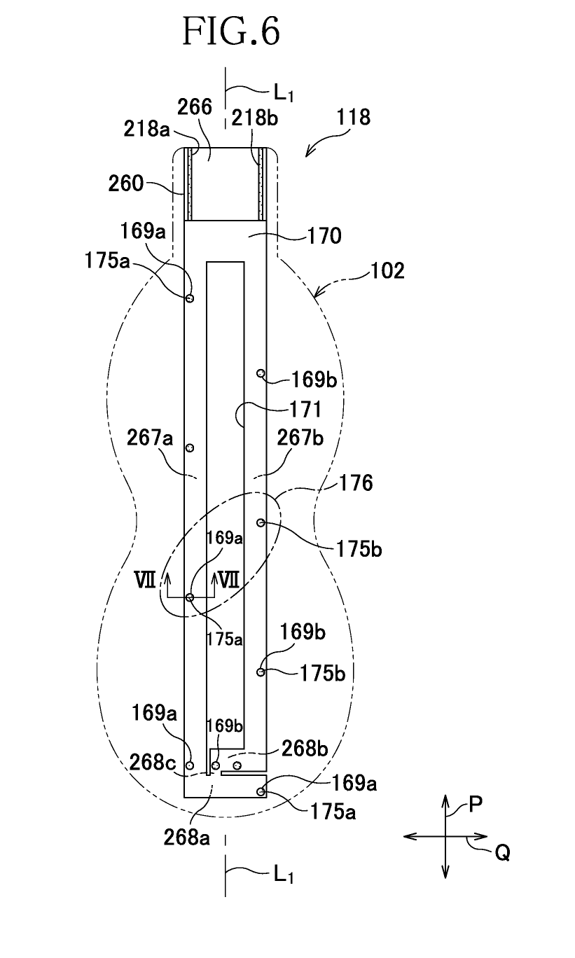

With the urine receiver 102 put on the wearer's body, if defecation occurs and the skin-contact sheet 134 is covered with feces, it will be difficult or sometimes impossible for the urine receiver 102 to detect urination and to start the suction of urine. To solve this problem, the urine receiver 102 is provided with a feces sensor 144 as will be apparent from FIGS. 3, 4 and 5. In the feces sensor 144, thermoplastic synthetic resin film sheets 142c, 142d are formed on respective upper surfaces with electrodes 142c, 142d and covered with cover sheets 142a, 142b, respectively. The cover sheet 142a is liquid-pervious and allows moisture in feces to pass therethrough toward the electrodes 143a, 143b. In this feces sensor 144, the electrodes 143a, 143b extend in the longitudinal direction P in parallel to the urine detecting electrodes 218a, 218b and have lower ends 145b as viewed in FIGS. 3 and 6 extend downward beyond the second end sheet 140. When the lower ends 145b are soiled with feces, moisture in feces permeates the cover sheet 142a, causing the electrodes 143a, 143b to be electrically connected to each other. Thereupon electric current is supplied from a power source 116a (See FIG. 1) included by the wiring 116 and an alarm device (not shown) in the pump unit 108 receives signals. In response to the signals, the alarm device prompts the care personnel to deal with feces and to exchange the urine receiver 102 with the fresh one.

FIG. 6 is a plan view of the urine sensor 118 used in the embodiment illustrated in FIGS. 3, 4 and 5 wherein the contour of the urine receiver 102 is indicated by imaginary line. The urine sensor 118 comprises a film sheet 260 formed of a synthetic resin film, a pair of the urine detecting electrodes 218a, 218b formed on one surface of the film sheet 260 and the insulating coating 170 covering the most part of these electrodes 218a, 218b. The film sheet 260 has a rectangular shape which is relatively long in the longitudinal direction P and formed in its middle in the transverse direction Q with a generally rectangular opening 171 largely cut out in the longitudinal direction P. Such film sheet 260 has an upper end section 266, as viewed in FIG. 6, adapted to be held by the clip 120, lateral sections 267a, 267b extending downward from the upper end section 266 on both sides of a central line L₁-L₁ bisecting a width dimension of the urine sensor 118, a lower end section 268a extending downward from the lower end of the lateral section 267a, and a lower end section 268b extending from the lower end section of the lateral section 267b. The lower end section 268a is connected with the lower end section 268b via a connecting region 268c. In the upper end section 266 of the film sheet 260, the electrodes 218a, 218b are exposed. In FIG. 6, a plurality of small circular regions in the lateral sections 267a, 267b and the lower end sections 268a, 268b indicate uncoated regions 169a, 169b formed in the insulating coating 170 in which a urine detecting region 175a or 175b of the electrode 218a or 218b are exposed.

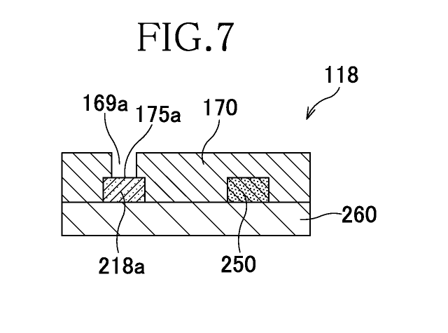

FIG. 7 is a sectional view taken along line VII-VII in FIG. 6. The film sheet 260 is formed on its upper surface with, in addition to the urine detecting electrode 218a, a resistive element 250 for detection of burnout and the urine detecting region 175a of the electrode 218a is exposed in the uncoated region 169a. The resistive element 250 is entirely covered with the insulating coating 170.

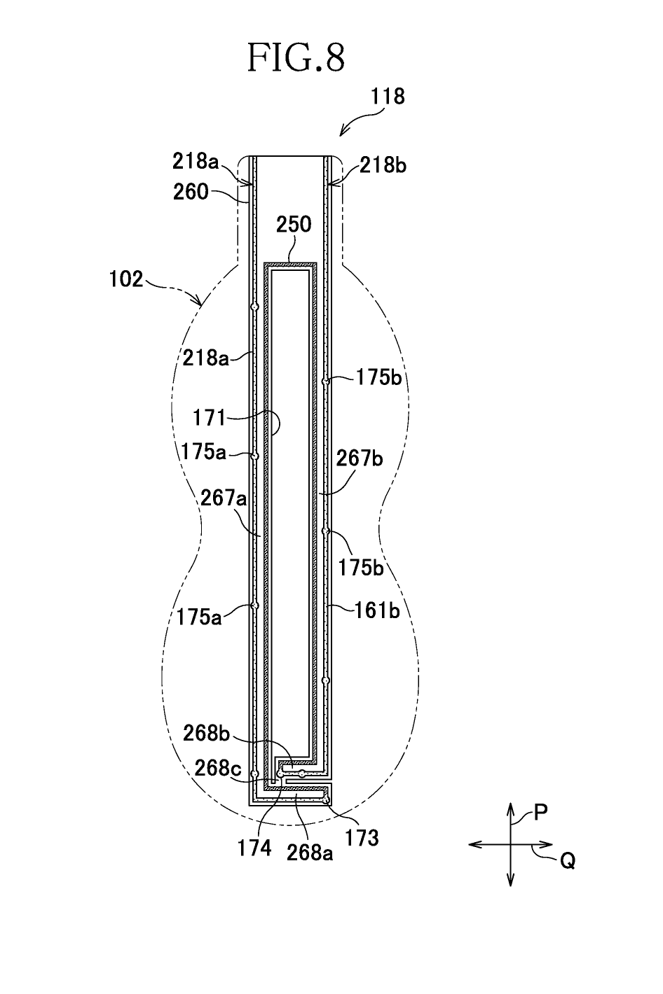

FIG. 8 is a plan view of the urine sensor 118 having the insulating coating peeled off. The film sheet 260 is formed on one surface with the generally L-shaped urine detecting electrode 218a extending along the lateral section 267a and the lower end section 268a and the inverted L-shaped urine detecting electrode 218b extending along the lateral section 267b and the lower end section 268b. The circular regions 175a, 175b of these electrodes 218a, 218b are exposed in the uncoated regions 169a, 169b shown in FIG. 6. The resistive element 250 for detection of burnout is formed between the electrodes 218a, 218b. The resistive element 250 is electrically connected to the respective lower end sections 173, 174 of the urine detecting electrodes 218a, 218b and extending along a peripheral edge of the opening 171.

As the film sheet 260 preferred to be used in such urine sensor 118, a polyester film having a thickness in a range of 50 to 100 μm may be used. The electrodes 218a, 218b may be obtained by printing the film sheet 260 with conductive ink or the other conductive coating material in a desired shape. The conductive ink or the conductive coating material may contain carbon black of 3 to 7% by mass, artificial graphite such as carbon graphite of 10 to 30% by mass and an appropriate quantity of silver powder. Each of the preferred electrodes 218a, 218b is designed to have a width dimension of 0.5 to 2 mm and a resistance value of 150KΩ or less and has the uncoated regions 169a or 169b each having a diameter of 1 to 2 mm. The burnout detecting resistance element 250 may be obtained by printing the film sheet 260 with ink, for example, containing carbon black of 3 to 7% by mass and artificial graphite of 5 to 10% by mass in a desired shape. This resistance element 250 has a resistance value substantially higher than that of the urine detecting electrodes 218a, 218b and the preferred resistance element 250 has a width dimension of 0.3 to 1 mm and a resistance value in the order of 2 to 10MΩ. If the kind of ink used to form the resistance element 250 has conductive properties as high as the type of ink used form the electrodes 218a, 218b has and the resistance element 250 is dimensioned to have a sufficiently large width to make the process easy, a total length of the resistance element 250 may be dimensioned as long as possible so as to extend in the longitudinal direction P along the peripheral edge of the opening 171 and thereby to increase the resistance value thereof.

In the case of the urine receiver 102 using the urine sensor 118 shown in FIG. 6, the wiring 116 including the power source 116a normally carries faint current A from the source 116a via the clip 120 to the electrodes 218a, 218b and the resistance element 250 electrically connected to these electrodes 218a, 218b. As long as an electric circuit 110 including the wiring 116 carries electric current A, the urine detecting electrodes 218a, 218b is determined to be in a normal state. In contrast, if no current A is detected, it is determined that failures such as burnout occur in the urine detecting electrodes 218a, 218b and signals prompting exchange of the urine sensor 118 is output from the pump unit 108. Referring to FIG. 6, when a range enclosed by an imaginary line 176 is wetted with urine, the urine flows into the non-coated regions 169a, 169b and comes in contact with the urine detecting regions 175a, 175b to put the electrode 218a extending along the lateral section 267a and the electrode 218b extending along the lateral section 267b into the electrically connected state. When a resistance value between the electrodes 218a, 218b becomes lower than a resistance value of the resistance element 250, the electric current no more flows through the resistance element 250 but flows through urine of which the resistance value is relatively low. When a value of the electric current B flowing through urine becomes higher than the electric current A flowing through the resistance element 250, the electric circuit 110 detects such change of the current or an abrupt change of voltage or resistance value and thereby to detect occurrence of urination. In response to signals generated on the basis of such change, the pump is actuated to suck urine and the pump is stopped as the resistance value between the electrodes 218a, 218b increases again.

While the non-coated regions 169a, 169b serving to expose the urine detecting regions 175a, 175b are formed in the lateral sections 267a, 267b asymmetrically about the center line L₁-L₁ in the urine sensor 118 shown in FIG. 6, it is also possible to form these non-coated regions 169a, 169b symmetrically about the center line L₁-L₁. In the lower end sections 268a, 268b of the film sheet 260 and in the vicinity of them, a plurality of the non-coated regions 169a, 169b narrowly spaced one from another. With the non-coated regions 169a, 169b arranged in this manner, even if urine immediately after discharged quickly spreads downward from above as viewed in FIG. 6 before reaching the air permeable-resistant sheet 124, the urine receiver 102 is capable of quickly sucking such urine. Alternatively, the non-coated regions 169a, 169b may be provided at appropriate position in the lateral sections 267a, 267b depending on the posture supposed to be taken by the wearer when the urine receiver 102 is put on the wearer's body.

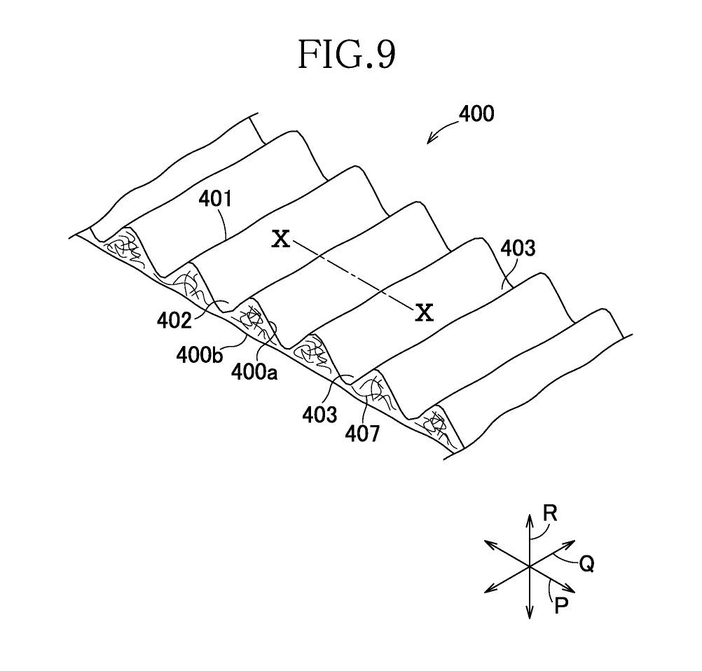

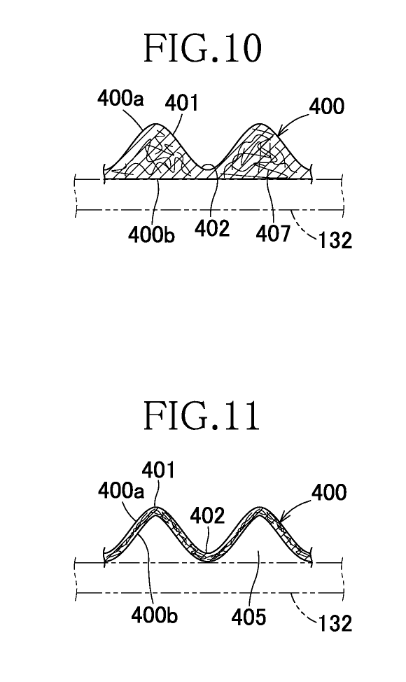

FIG. 9 is a perspective view of a sheet 400 as an example of the skin-contact sheet 134 and FIG. 10 is a sectional view taken along line X-X in FIG. 9. The sheet 400 is formed of a nonwoven fabric containing hydrophobic thermoplastic synthetic fibers of 80 to 100% by mass and liquid-absorbent fibers such as rayon fibers of 20 to 0% by mass wherein these fibers are fusion-bonded or interlaced together. The sheet 400 has an outer surface 400a facing the wearer's skin and an inner surface 400b facing the urine sensor 108 wherein a longitudinal direction, a transverse direction and a thickness direction correspond to the longitudinal direction P, the transverse direction Q and the thickness direction R of the urine receiver 102, respectively. The outer surface 400a and the inner surface 400b correspond to the upper surface and the lower surface, respectively, in FIG. 4.

The outer surface 400a of the sheet 400 has undulation formed of crests 401 and troughs 402 alternating in the longitudinal direction P and the crests 401 and the troughs 402 extending in the transverse direction Q in parallel one to another. The inner surface 40b of the sheet 400 is flat and put in close contact with the filter 132. The sheet 400 is used in a manner that the crests 401 and the troughs 402 may extend across a pair of the electrodes 218a, 218b in the urine sensor 118 and side edges 403 opposed in the transverse direction Q may be preferably located inside the respective leakage-barriers 136 (See FIG. 4) provided on both sides of the urine receiver 102.

With the urine receiver 102 using the sheet 400 as the skin-contact sheet 134 and put on the wearer's body, the urine discharged toward the sheet 400 is apt to be collected in the troughs 402 and then to spread in the transverse direction Q. In the urine receiver 102, therefore, the paired electrodes 218a, 218b can be quickly connected via urine and thereupon the urine receiver 102 can start to suck the discharged urine. The urine receiver 102 utilizing function of the troughs 402 of the sheet 400 is advantageous particularly in that, even when the wearer is in the recumbent position, the discharged urine spreads in the transverse direction Q and the paired electrodes 218a, 218b can be quickly connected via urine.

The urine detecting regions 175a, 175b (See FIG. 6) in the electrodes 218a, 218b used in combination with the sheet 400 functioning in the manner as described above are preferably formed symmetrically about the center line L₁-L₁. The sheet 400 has a basis mass in a range of 15 to 80 g/m² and comprises a plurality of hydrophobic thermoplastic synthetic fibers 407 each having a fineness in a range of 1 to 8 dtex and a fiber length in a range of 20 to 80 mm wherein most of the thermoplastic synthetic fibers 407 are preferably oriented in the transverse direction Q. Such sheet 400 can be obtained by heating and pressing a nonwoven fabric such as a thermally bonded nonwoven fabric between vertically paired molds wherein the upper mold has the crests and the troughs and the lower mold is flat. A machine direction in the course of producing the nonwoven fabric may be aligned with the direction in which the troughs extend to orient the thermoplastic synthetic fibers in the transverse direction Q. The sheet 400 may be previously treated to become more hydrophilic in the troughs 402 than in the crests 401 to promote the movement of the discharged urine from the crests 401 to the troughs 402. If desired, it is also possible to make the sheet 400 so that respective tops of the crests 401 are water-repellent and respective bottoms of the troughs 402 are hydrophilic.

FIG. 11 is a view similar to FIG. 10, exemplarily showing the sheet 400 which is different from those shown in FIGS. 9 and 10. The sheet 400 shown in FIG. 11 has a generally uniform thickness over its entire area, i.e., the inner surface 400b extends in parallel to the outer surface 400a, and has crests and troughs 411, 412 corresponding to the those 401, 402 of the sheet shown in FIGS. 9 and 10. The inner surface 400b are intermittently bonded or heat-sealed along respective bottoms of the troughs 412 to the filter 132 facing the inner surface 400b of the sheet 400. In this manner, the sheet 400 is joined to the filter 132 intermittently in the transverse direction Q and the sheet 400 and the filter 132 are put in close contact with each other in a region defined between each pair of the adjacent joints so as to facilitate passage of urine through the sheet 400 and the filter 132. Tunnels 405 extending in the transverse direction Q are defined between the sheet 400 and the filter 132 and therefore the urine receiver 102 having a feature shown in FIG. 11 assures an air-permeability higher than the urine receiver 102 having a feature shown in FIGS. 9 and 10. The sheet 400 exemplarily shown in FIG. 11 may be obtained by sandwiching a nonwoven fabric such as a thermally bonded nonwoven fabric between each pair of heated molds from above and below. These paired molds may have the crests and troughs, respectively, adapted to be engaged one with another.

The sheet exemplarily shown in FIGS. 10 and 11 is used not only as the skin-contact sheet 134 but also used so as to lie between the skin-contact sheet 134 and filter 132 in FIG. 4. When the fiber interstice in the sheet 400 is comparable to the fiber interstice in the filter 132, the filter 132 may be replaced by the sheet 400. In any case, the sheet 400 lies outside the electrodes 218a, 218b in the depth direction R and the crests 401 and the troughs 402 extend across these electrodes 218a, 218b. With such an arrangement, the urine receiver 102 allows these electrodes 218a, 218b to be electrically connected with each other as quickly as possible. It should be appreciated that the dispersant sheet 126 may be eliminated if the air permeable-resistant sheet 124 contains, in addition to the thermoplastic synthetic fibers, hydrophilic fiber such as rayon fiber and thereby promotes dispersion of urine.

Details of the present invention on the second aspect thereof will be more fully understood from the description exemplarily on the basis of pants formed of a mesh sheet as a wearing article incorporated with the urine receiver.

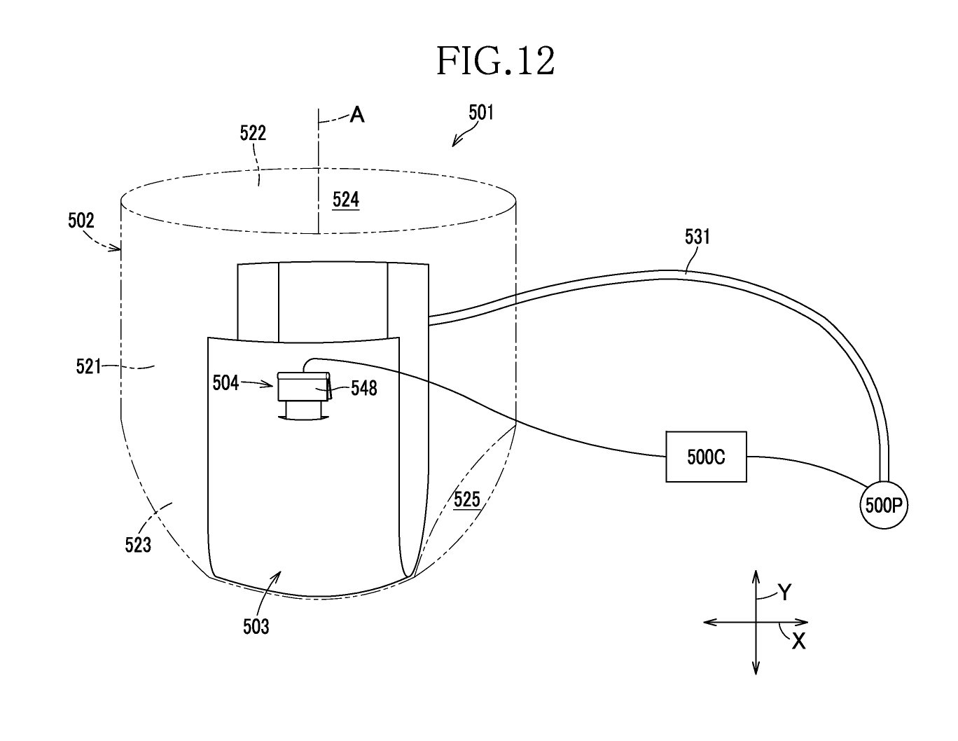

FIG. 12 is an overall diagram schematically illustrating the wearing article 501 according to the present invention. According to the present invention, a pants-type chassis 502 is formed therein with a urine receiver 503 to which a suction pump 500P as a suction unit via suction tube 531. The urine receiver 503 is electrically connected to the suction pump 500P which is adapted to be actuated by a control unit 500C when a moisture detecting structure 504 incorporated in the urine receiver 503 detects occurrence of urination. Upon actuation of the suction pump 500P, the urine collected by the urine receiver 503 is sucked and then evacuated out from the chassis 502. In FIG. 12, the chassis 502 is indicated by imaginary lines.

The chassis 502 comprises a front waist region 521, a rear waist region 522 and a crotch region 523 extending between these front and rear waist regions 521, 522 wherein these three regions are continuous in the longitudinal direction Y. The chassis 502 is formed of an elastic mesh sheet so that the chassis 502 may be elasticized in the longitudinal direction Y and in the transverse direction X which is orthogonal to the longitudinal direction Y. Particularly along peripheries of a waist-opening 524 and leg-openings 525, the elasticity is locally heightened to improve a fit of the chassis 502 to the wearer's waist and thighs, respectively.

On the side of the chassis 502 facing the wearer's body, the urine receiver 503 extends in the longitudinal direction Y from the front waist region 521 to the rear waist region 522 along a longitudinal center line A-A bisecting a dimension of the chassis 502 in the transverse direction X. The urine receiver 503 is kept in close contact with the wearer's body under the effect of elasticity of the chassis 502 so that only the urine receiver 503 may be exchanged as the need arises.

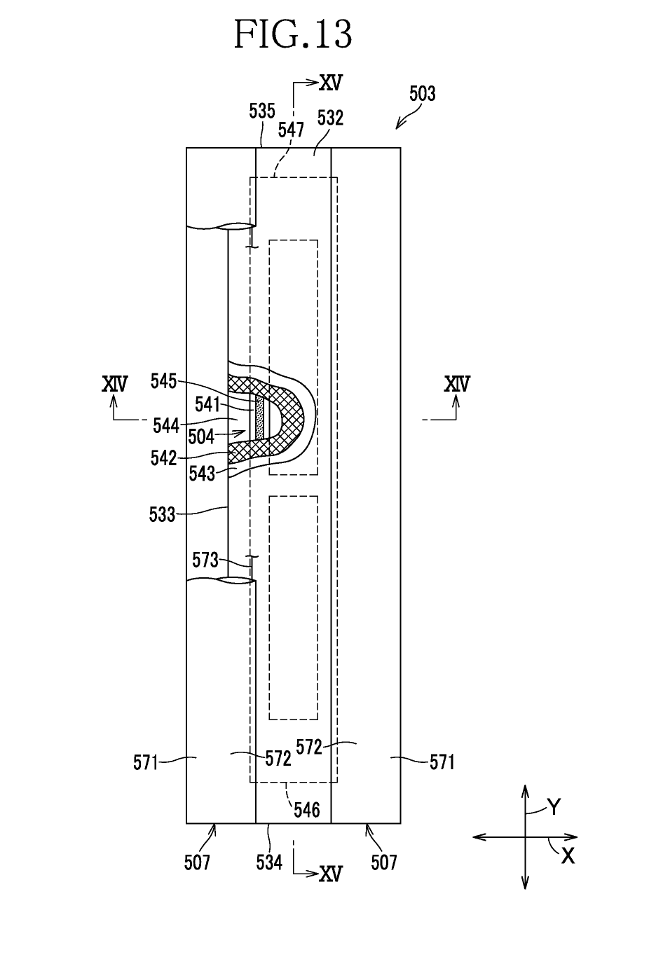

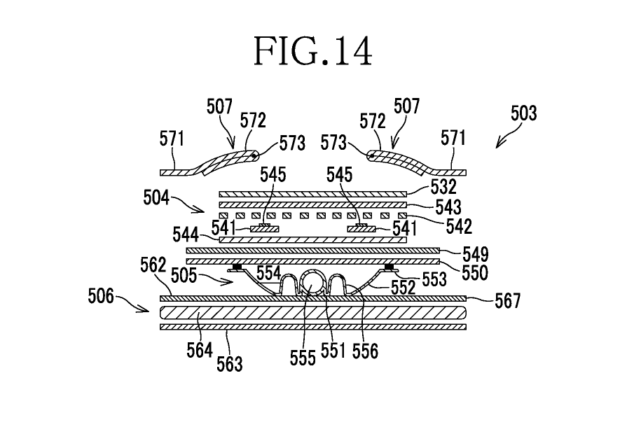

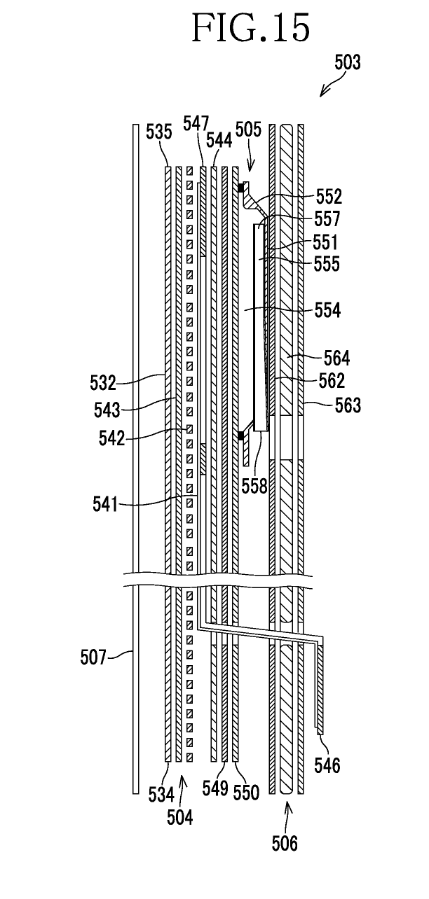

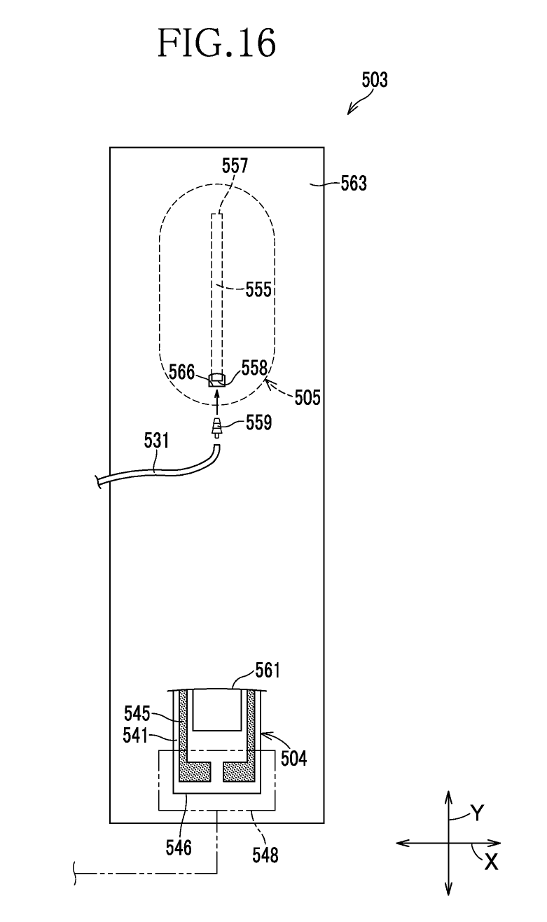

FIG. 13 is a plan view of the urine receiver 503 as viewed from its side facing the wearer's body. It should be appreciated that the urine receiver 503 is shown as partially cutaway and the chassis 502 is not shown at all for convenience of explanation. FIG. 14 is a sectional view taken along line XIV-XIV in FIG. 13 and FIG. 15 is a sectional view taken along a line XV-XV in FIG. 13. While the respective sheets are illustrated to be spaced one from another for convenience of explanation in FIGS. 14 and 15, these sheets are normally joined together. FIG. 16 is a plan view of the urine receiver of FIG. 13 as viewed from the side facing the wearer's garment.

The urine receiver 503 comprises a topsheet 532 adapted to come in contact with the wearer's body, a moisture detecting structure 504 serving to detect urination, a tray-shaped urine receptacle 505 located on the side of the moisture detecting structure 504 facing the wearer's body to receive discharged urine and a moisture absorbent structure 506 lying on the side of the urine receptacle 505 facing the wearer's garment to absorb moisture vapor in the chassis 502.

The topsheet 532 may be formed, for example, of a liquid-pervious fibrous nonwoven fabric. The topsheet 532 has a generally rectangular shape extending in the longitudinal direction Y and includes opposite side edges 533, 533 extending in the longitudinal direction Y and front and rear ends 534, 535 extending in the transverse direction X.

The moisture detecting structure 504 extends the front waist region 521 to the rear waist region 522 of the chassis 502. The moisture detecting structure 504 comprises a pair of moisture detecting sensors 541, a spacer 542 lying on the side of the moisture detecting sensors 541 facing the wearer's body, a filter 543 lying on the side of the spacer 542 facing the wearer's body and a cushion sheet 544 lying on the side of the moisture detecting sensors 541 facing the wearer's garment. Specifically, the moisture detecting sensors 541 is sandwiched between the spacer 542 and the cushion sheet 544 in the thickness direction and thereby protected from any external shock.

The moisture detecting sensors 541 are formed of a pair of electrodes 545, 545 spaced from each other in the transverse direction X and these electrodes 545, 545 are obtained, for example, by printing a thermoplastic synthetic film such as a polyethylene film with a conductive coating material. These electrodes 545, 545 extend in parallel to each other in the longitudinal direction Y. The moisture detecting sensors 541 respectively have front and rear ends 546, 547 extending in the transverse direction X wherein the sensor's front end 546 extends through a slit 561 formed in the moisture absorbent structure 506 to the side thereof facing the wearer's garment. The slit 561 is formed on the side of the front waist region 521. The sensor's front end 546 extending out through the slit 561 is provided with a clip 548 via which the signal from the moisture detecting sensors 541 is transmitted to the control unit 500C.

More specifically, the moisture detecting sensors 541 are turned on in response to electrical connection of the paired electrodes 545, 545 effectuated by the present of urine therebetween and the control unit 500C detects occurrence of urination on the basis of this turned-on state.

As a material for the spacer 542, a mesh sheet having high air- and liquid-permeability and serves to keep the filter 543 and the moisture detecting sensors 541 spaced from each other in the thickness direction. If the filter 543 in a wet condition continues to be kept in contact with the moisture detecting sensors 541, the moisture detecting sensors 541 will be maintained in the turned-on state and may malfunction to detect urination even though no urination occurs. To prevent such malfunction, the filter 543 and the moisture detecting sensors 541 are kept spaced from each other. For the purpose of preventing such malfunction, use of the spacer is particularly effective since the spacer can effectively prevent these filter 543 and moisture detecting sensors 541 from being continuously kept in contact with each other.

A dispersant sheet 549 and an air permeable-resistant sheet 550 are stacked on the side of the cushion sheet 544 facing the wearer's garment and the urine receptacle 505 is formed on the side of the air permeable-resistant sheet facing the wearer's garment. The air permeable-resistant sheet 550 and the urine receptacle 505 are joined together, for example, by adhesives.

The dispersant sheet 549 serves to disperse the discharged urine as quickly as possible and thereby to change the air permeable-resistant sheet 550 over a wide range thereof into a dry condition. As a material for the air permeable-resistant sheet 549, for example, a nonwoven fabric containing hydrophilic fibers such as rayon fibers may be used.

The air permeable-resistant sheet 550 is high in its liquid-permeability but low in its air-permeability and, as a material for this sheet 550, for example, a nonwoven fabric modified to become hydrophilic may be used.

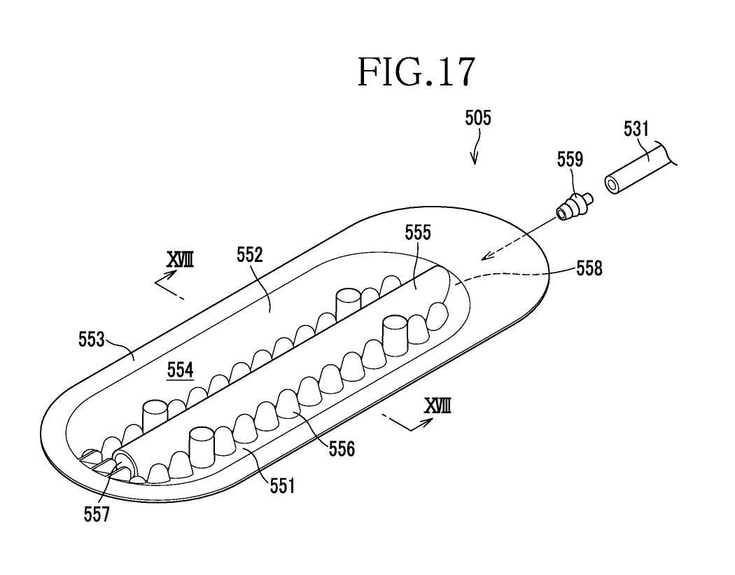

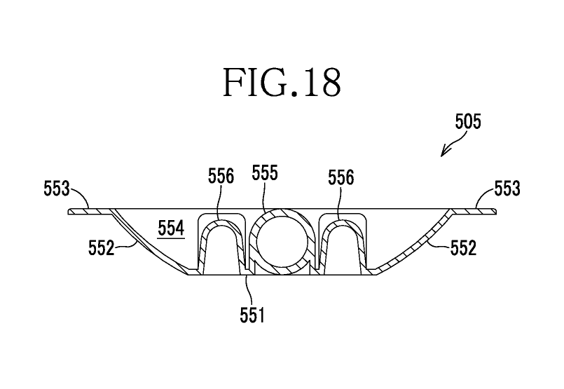

The urine receptacle 505 is flexible and may be formed, for example, of a liquid-impervious soft elastic material such as a soft polyethylene or silicon rubber. FIG. 17 is a perspective view of the urine receptacle 505 and FIG. 18 is a sectional view taken along line XVIII-XVIII in FIG. 17. The urine receptacle 505 comprises a bottom 551, a peripheral wall 552 rising from the bottom 551 toward the side of the wearer's body, and a flange 553 extending outward from a top peripheral edge of the peripheral wall 552. The flange 553 is put in contact with the air permeable-resistant sheet 550 to form a space 554 within the urine receptacle 505. The urine receptacle 505 is formed with a suction unit 555 functioning to collect the urine flowing thereinto and to evacuate this out of the urine receptacle 505. The suction unit 555 has a cylindrical shape and extends along a generally transverse middle zone of the receptacle 505 in the longitudinal direction. On both sides of the suction unit 555, a plurality of protuberances 556 extend upward from the bottom 551 toward the wearer's body. These protuberances 556 serve to prevent the air permeable-resistant sheet 550 which should be held in contact with the flange 553 from sagging down into the space 554.

Referring to FIG. 16, the suction unit 555 has one end 557 opening into the urine receptacle 505 and the other end 558 opening to the outside of the urine receptacle 505. The suction tube 531 is attached to the other end 558 of the suction unit 555 with a joint 559 and the suction tube 531 is connected to the suction pump 500P.

The suction tube 531 and the suction pump 500P both connected to the other end 558 of the suction unit 555 define a moisture suction device in the present invention. Upon actuation of the suction pump 500P, the urine collected in the urine receptacle 505 is sucked through the suction tube 531. The suction pump 500P is electrically connected to the control unit 500C so that the control unit 500C detects occurrence of urination on the basis of signals transmitted from the moisture detecting sensors 541 and thereupon transmits signals to the suction pump 500P. In response to the signals from the control unit 500C, the suction pump 500P is actuated. In this way, the suction pump 500P is actuated only when the urination occurs.

Referring to FIGS. 14 and 15, the moisture absorbent structure 506 is formed on the side of the urine receptacle 505 facing the wearer's garment. The moisture absorbent structure 506 comprises a moisture-pervious sheet 562 lying on the side facing the wearer's body, a liquid-impervious sheet 563 lying on the side facing the wearer's garment and moisture absorbent material 564 lying between these two sheets 562, 563. The moisture absorbent material 564 comprises a moisture-absorbent core material such as fluff pulp, superabsorbent polymer particles (SAP) or silica gels wrapped with tissue paper or the like. As a material for the moisture-pervious sheet 562, for example, a thermoplastic drawn film containing inorganic fillers may be used. As the inorganic fillers, calcium carbonate, barium sulfate or the like and, as the thermoplastic film, polyethylene films or the like may be used. As both the inorganic fillers and the thermoplastic films, those widely used in the relevant technical field may be used. Moisture permeability of the moisture-pervious sheet 562 is preferably in a range of 83.3 to 166.6 (g/m²/24 hours). If the moisture permeability is lower than 83.3 (g/m²/24 hours), a significant moisture absorbing effect will not be expressed and, if the moisture permeability is higher than 166.6 (g/m²/24 hours), urine will directly infiltrate into the moisture-absorbent material. In this case, there is a possibility that the urine infiltrating into the moisture-absorbent material may cause a temperature within the wearing article to rise.

Moisture permeability was measured in accordance with JIS-Z-0208. Specifically, circular pieces each having a diameter of 0.03 m were cut out from the moisture-pervious sheet as test pieces defining the boundary plane for moisture permeation. A thermo-hygrostat was set to a temperature of 40° C. and a humidity of 60%. Total mass (g) of moisture vapor had passed through this boundary plane during 24 hours was converted to the mass per 1 m² of the tested sheet to obtain the moisture permeability.

The moisture permeability was calculated by an equation as follows:

Permeability (Pa)=mass (g) before measurement−mass (g) 24 hours after starting of measurement/π×0.03×0.03/24 hours.

The moisture-absorbent structure 506 has a length dimension in the transverse direction X larger than that of the moisture detecting structure 504 lying on the side of the moisture-absorbent structure 506 facing the wearer's body. In other words, the moisture-absorbent structure 506 extends in the transverse direction X beyond the both ends of the moisture detecting structure 504. In the sections extending beyond the both ends of the moisture detecting structure 504, a pair of barrier cuffs 507, 507 against body waste is formed on the side of the moisture-pervious sheet 562 facing the wearer's body constituting the moisture-absorbent structure 506.

The barrier cuffs 507, 507 are attached to side edges 567, 567 of the moisture-pervious sheet, respectively, and include proximal edges 571, 571 joined to the side edges 567, 567 of the moisture-pervious sheet 562 and free edges 572, 572 lying inside of the proximal edges 571, 571 in the transverse direction X and not joined to the moisture-pervious sheet 562. These free edges 572, 572 lie on the side of the topsheet 632 facing the wearer's body and provided with cuff elastic elements 573, 573 extending in the longitudinal direction Y and attached thereto under tension and in a contractible manner. Upon contraction of the cuff elastic elements 573, 573, the free edges 572, 572 are spaced upward so as to stand on the topsheet 532 and thereby prevent urine from leaking outward in the transverse direction X. While the liquid-impervious barrier cuffs 507, 507 are effective to prevent leakage of urine, it is possible to use, for example, moisture-pervious fibrous nonwoven fabric from the viewpoint of permeability for moisture vapor.

Now the manner in which the aforementioned arrangement operates in response to occurrence of urination will be described. Upon occurrence of urination, the urine discharged infiltrates into the topsheet 532. The barrier cuffs 507, 507 formed along the side edges 533, 533 of the topsheet prevent the urine from leaking outward in the transverse direction X.

The urine having infiltrated into the topsheet 532 quickly come in contact with the moisture detecting sensors 541 through the spacer 542 and the filter 543. When urine comes in contact with the paired moisture detecting sensors 541 so as to connect these sensors with each other, these moisture detecting sensors 541 are turned on and transmit signals to the control unit 500C. In response to this signal, the control unit 500C actuates the suction pump 500P. Upon actuation of the suction pump 500P, the space 554 of the urine receptacle 505 is subjected to a suction effect via a suction unit 555.

The urine having reached the moisture detecting sensor 541 infiltrates into the dispersant sheet 549 through the cushion sheet 544. The urine having dispersant sheet 549 over a wide range simultaneously infiltrates into the air permeable-resistant sheet 550 which covers the opening of the urine receptacle 505 to define the space 554 therein. When the space 554 is subjected to the suction effect by the suction pump 500P and the opening is covered with the air permeable-resistant sheet 550 wetted with urine, a negative pressure is generated within the space 554. As a result, the urine having infiltrated in the air permeable-resistant sheet 550 is sucked into the space 554 and the urine sucked into the space 554 is sucked through the suction tube 555 from the urine receptacle 505. In this way, immediately after occurrence of urination, the discharged urine can be quickly evacuated out of the wearing article.

After the discharged urine has sucked and evacuated, substantially entire quantity of urine is evacuated from the wearing article but there is a possibility that a small quantity of urine might be left, for example, in the urine receiver 503. Particularly because the urine receptacle 505 is liquid-impervious, the possibility that a small quantity of urine might be left on its side facing the wearer's body is correspondingly high. The urine left not sucked will be vaporized by the wearer's body heat and will pervade the interior of the wearing article. However, the moisture-absorbent structure 506 formed on the side of the urine receiver 503 facing the wearer's garment is effective to absorb such moisture vapor.

Specifically, moisture vapor passes through the moisture-pervious sheet 562 of the moisture-absorbent structure 506 and is absorbed by the moisture-absorbent material 564. The liquid-impervious sheet 563 lies on the side of the moisture-absorbent material 564 and therefore moisture vapor is reliably absorbed by the moisture-absorbent material 564 without leaking out. Even if moisture vapor is generated within the wearing article 501, such moisture vapor can be reliably absorbed by the moisture-absorbent material 564. In consequence, there is no possibility that a temperature within the pants might rise and the wearer might suffer from any skin troubles or a discomfortable feeling due to the undesirable humidity.

The moisture absorbent structure 506 is dimensioned in the transverse direction X to be larger than that of the moisture detecting structure 504. With such dimensioning, moisture vapor can be absorbed by the section extending outward beyond the periphery of the urine receiver 503 and therefore the absorbing function should not be disturbed by various sheets of the urine receiver 503.

Even if moisture vapor within the wearing article 501 increases due not only urine but also to the wearer's sweat of the wearer, the moisture-absorbent structure 506 can effectively absorb moisture vapor and thereby restrict increase of temperature.

While the wearer's article is provided with the moisture detecting sensor 541 serving for detection of urine according to the present embodiment, it is possible to incorporate, in addition to the moisture detecting sensor 541, a feces detecting sensor. While the mesh pants are used as the chassis 502 according to the present embodiment, it is also possible to use widely used means such as disposable diapers or diaper covers as the chassis 502.

Details of the present invention on a third aspect thereof will be more fully understood from the description exemplarily on the basis of pants formed of a mesh sheet as a wearing article incorporated with the urine receiver.

### First Embodiment

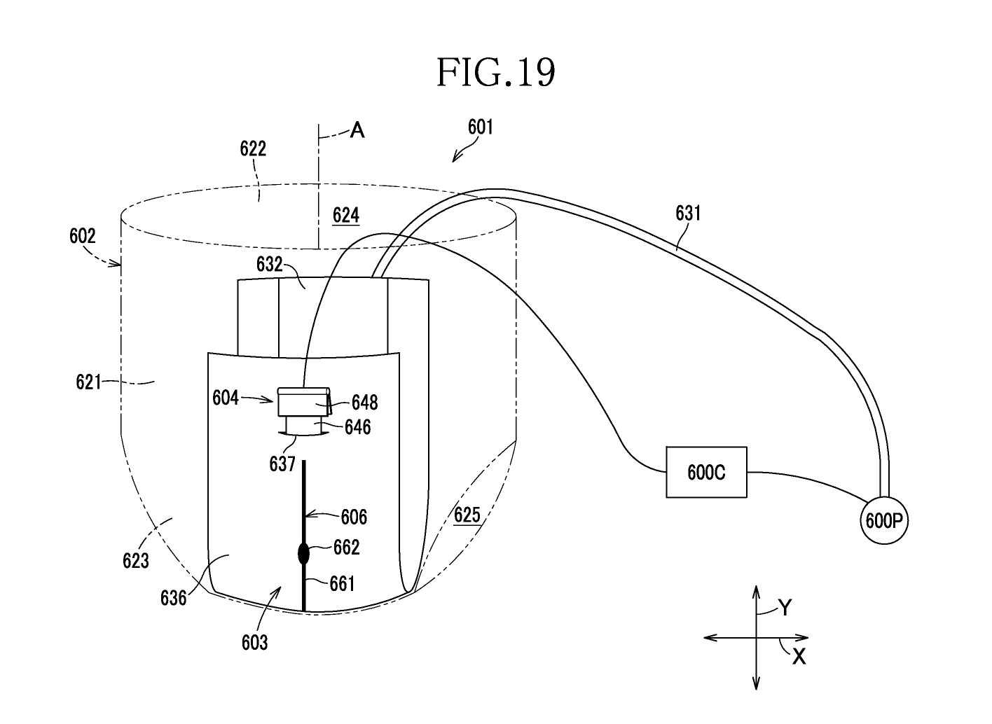

FIGS. 19 through 24 illustrate a first embodiment of the present invention on the third aspect thereof.

FIG. 19 is an overall diagram schematically illustrating the wearing article 601 according to the present invention. According to the present invention, a pants-type chassis 602 is formed therein with a urine receiver 603 to which a suction pump 600P as a suction unit via suction tube 631. The urine receiver 603 is electrically connected to the suction pump 600P which is adapted to be actuated by a control unit 600C when a moisture detecting structure 604 incorporated in the urine receiver 603 detects occurrence of urination. Upon actuation of the suction pump 600P, the urine collected by the urine receiver 603 is sucked and then evacuated out from the chassis 602. In FIG. 19, the chassis 602 is indicated by imaginary lines.

The chassis 602 comprises a front waist region 621, a rear waist region 622 and a crotch region 623 extending between these front and rear waist regions 621, 622 wherein these three regions are continuous in the longitudinal direction Y. The chassis 602 is formed of an elastic mesh sheet so that the chassis 602 may be elasticized in the longitudinal direction Y and in the transverse direction X which is orthogonal to the longitudinal direction Y. Particularly along peripheries of a waist-opening 624 and leg-openings 625, the elasticity is locally heightened to improve a fit of the chassis 602 to the wearer's waist and thighs, respectively.

In the chassis 602, the urine receiver 603 can be visually recognized through the elastic mesh formed of elastic yarns.

The receiver 603 extends in the longitudinal direction Y from the front waist region 621 to the rear waist region 622 and along a longitudinal center line A-A bisecting a dimension in the transverse direction X. The receiver 603 is kept in close contact with the wearer's body under elastic force of the chassis 602 so that the receiver can be independently exchanged with the fresh one.

The receiver 603 includes the topsheet 632 lying on the side facing the wearer's body and the backsheet 636 facing the wearer's garment wherein the backsheet 636 is formed with a linear alignment check mark 606 extending in the longitudinal direction Y.

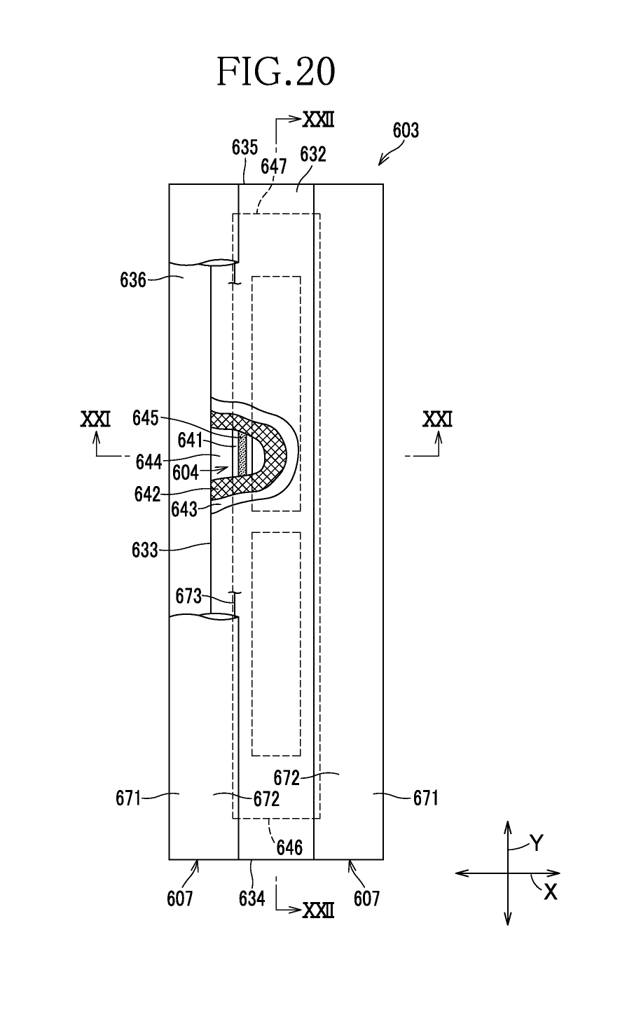

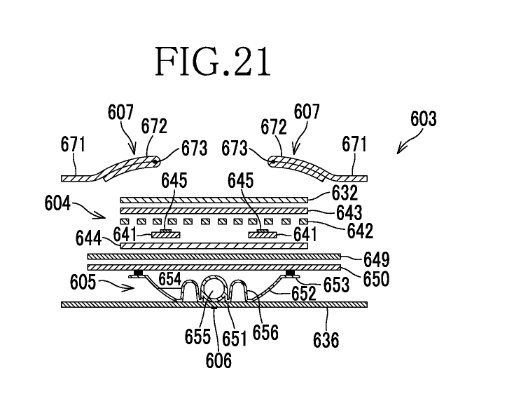

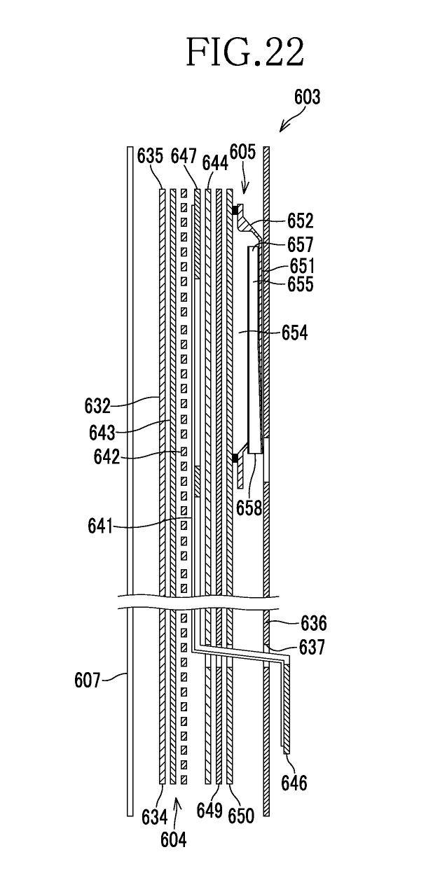

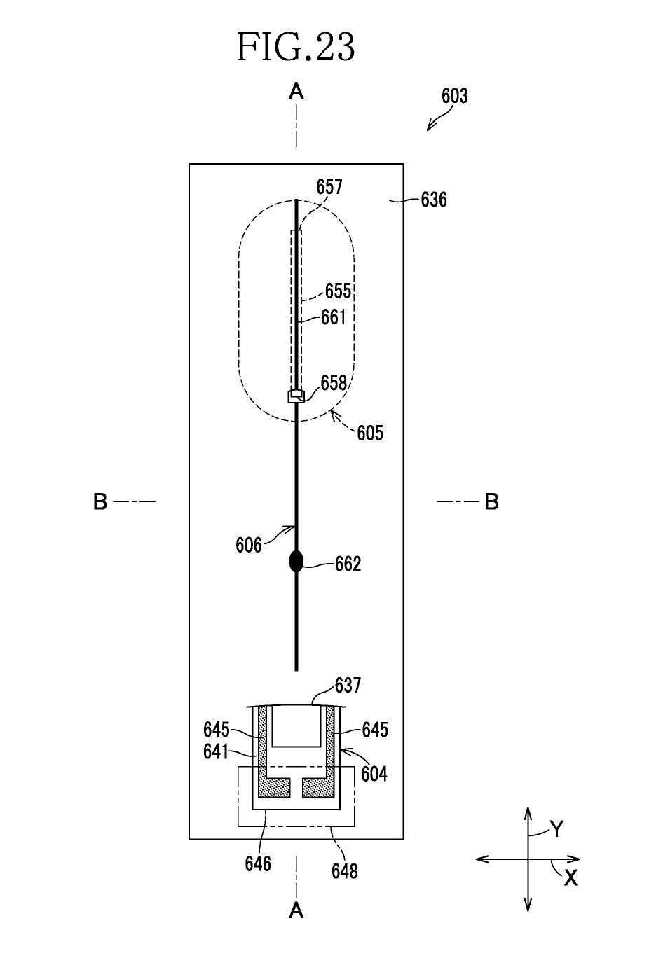

FIG. 20 is a plan view showing the receiver 603 in its developed state and as viewed from the side of the wearer's body. In FIG. 20, the receiver 603 is shown as partially cutaway and the chassis 602 is not shown for convenience of explanation. FIG. 21 is a sectional view taken along line XXI-XXI in FIG. 20 and FIG. 22 is a sectional view taken along line XXII-XXII in FIG. 20. In FIGS. 21 and 22, the respective sheets are shown as being spaced one from another for convenience of explanation but these sheets are normally joined together. FIG. 23 is a plan view showing the receiver 603 as viewed from the side of the wearer's garment.

A pair of barrier cuffs 607, 607 against body waste is formed on the side of the topsheet 632 facing the wearer's body. The barrier cuffs 607, 607 respectively include proximal edges 671, 671 lying on outer sides as viewed in the transverse direction X and free edges 672, 672 lying on the inner sides as viewed in the transverse direction X. The proximal edges 671, 671 extend outward in the transverse direction X beyond the topsheet 632 and joined to the backsheet 636. The free edges 672, 672 are adapted to be spaced upward from the topsheet 632 toward the wearer's body and provided with cuff elastic elements 673, 673 extending in the longitudinal direction Y and attached thereto under tension and in a contractible manner. Upon contraction of the cuff elastic elements 673, 673, the free edges 672, 672 are spaced upward so as to stand on the topsheet 632 and thereby prevent urine from leaking outward in the transverse direction X. While the liquid-impervious barrier cuffs 607, 607 are effective to prevent leakage of urine, it is possible to use, for example, a moisture-pervious fibrous nonwoven fabric from the viewpoint of permeability for moisture vapor.

Between the top- and backsheets 632, 636, the moisture detecting structure 604 serving to detect occurrence of urination and the tray-shaped urine receptacle 605 lying on the side of the moisture detecting structure 604 facing the wearer's garment and adapted to receive discharged urine.

The moisture detecting structure 604 extends the front waist region 621 to the rear waist region 622 of the chassis 502. The moisture detecting structure 604 comprises a pair of moisture detecting sensors 641, a spacer 642 lying on the side of the moisture detecting sensors 641 facing the wearer's body, a filter 643 lying on the side of the spacer 642 facing the wearer's body and a cushion sheet 644 lying on the side of the moisture detecting sensors 641 facing the wearer's garment. Specifically, the moisture detecting sensors 641 is sandwiched between the spacer 642 and the cushion sheet 644 in the thickness direction and thereby protected from any external shock.

The moisture detecting sensors 641 are formed of a pair of electrodes 645, 645 spaced from each other in the transverse direction X and these electrodes 645, 645 are obtained, for example, by printing a thermoplastic synthetic film such as a polyethylene film with conductive coating material. These electrodes 645, 645 extend in parallel to each other in the longitudinal direction Y. The moisture detecting sensors 641 respectively have front and rear ends 646, 647 extending in the transverse direction X wherein the sensor's front end 646 extends through a slit 637 formed in the backsheet 636 to the side thereof facing the wearer's garment. The slit 637 is formed on the side of the front waist region 621. The sensor's front end 646 extending out through the slit 637 is provided with a clip 648 via which the signal from the moisture detecting sensors 641 is transmitted to the control unit 600C.

More specifically, the moisture detecting sensors 641 are turned on in response to electrical connection of the paired electrodes 645, 645 effectuated by the present of urine therebetween and the control unit 600C detects occurrence of urination on the basis of this turned-on state.

As a material for the spacer 642, a mesh sheet having high air- and liquid-permeability and serves to keep the filter 643 and the moisture detecting sensors 641 spaced from each other in the thickness direction. If the filter 643 in a wet condition continues to be kept in contact with the moisture detecting sensors 641, the moisture detecting sensors 641 will be maintained in the turned-on state and may malfunction to detect urination even though no urination occurs. To prevent such malfunction, the filter 643 and the moisture detecting sensors 641 are kept spaced from each other. For the purpose of preventing such malfunction, use of the spacer is particularly effective since the spacer can effectively prevent these filter 643 and moisture detecting sensors 641 from being continuously kept in contact with each other.

A dispersant sheet 649 and an air permeable-resistant sheet 650 are stacked on the side of the cushion sheet 644 facing the wearer's garment and the urine receptacle 605 is formed on the side of the air permeable-resistant sheet facing the wearer's garment. The air permeable-resistant sheet 650 and the urine receptacle 605 are joined together, for example, by adhesives.

The dispersant sheet 649 serves to disperse the discharged urine as quickly as possible and thereby to change the air permeable-resistant sheet 650 over a wide range thereof into a dry condition. As a material for the air permeable-resistant sheet 649, for example, a nonwoven fabric containing hydrophilic fibers such as rayon fibers may be used.

The air permeable-resistant sheet 650 is high in its liquid-permeability but low in its air permeability and, as a material for this sheet 650, for example, a nonwoven fabric modified to become hydrophilic may be used.

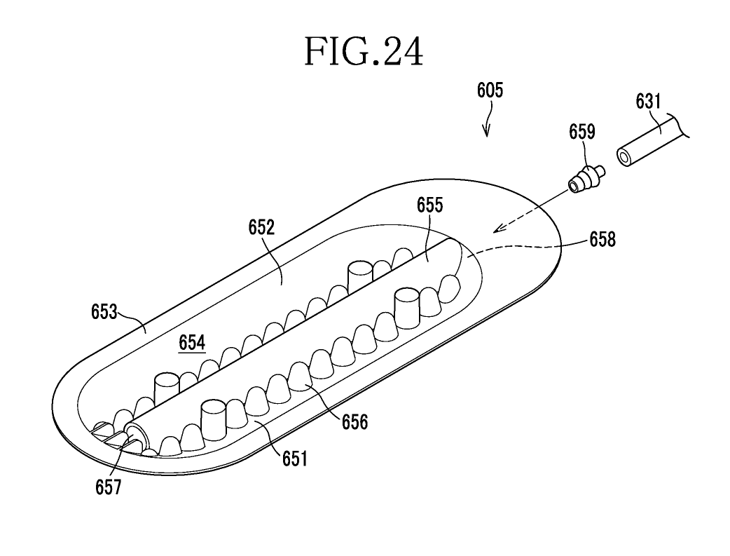

The urine receptacle 605 is flexible and may be formed, for example, by a liquid-impervious soft elastic material such as soft polyethylene or silicon rubber. FIG. 24 is a perspective view of the urine receptacle 605. The urine receptacle 605 comprises a bottom 651, a peripheral wall 652 rising from the bottom 651 toward the side of the wearer's body, and a flange 653 extending outward from a top peripheral edge of the peripheral wall 652. The flange 653 is put in contact with the air permeable-resistant sheet 650 to form a space 654 within the urine receptacle 605. The urine receptacle 605 is formed with a suction unit 655 functioning to collect the urine flowing thereinto and to evacuate this out of the urine receptacle 605. The suction unit 655 has a cylindrical shape and extends along a generally transverse middle zone of the receptacle 605 in the longitudinal direction. On both sides of the suction unit 655, a plurality of protuberances 656 extend upward from the bottom 651 toward the wearer's body. These protuberances 656 serve to prevent the air permeable-resistant sheet 650 which should be held in contact with the flange 653 from sagging down into the space 654.

The suction unit 655 has one end 657 opening into the urine receptacle 605 and the other end 658 opening to the outside of the urine receptacle 605. The suction tube 631 is attached to the other end 658 of the suction unit 655 with a joint 659 and the suction tube 631 is connected to the suction pump 600P.

The backsheet 636 lies on the side of the urine receiver 605 facing the wearer's garment. The backsheet 636 may be formed, for example, of a liquid-impervious fibrous nonwoven fabric. On the side of the backsheet 636 facing the wearer's garment, the alignment check mark 606 is formed (See FIGS. 19 and 23). The alignment check mark 606 comprises a linear first segment 661 extending in the longitudinal direction Y and a second segment 662 having a dimension in the transverse direction X larger than that of the first segment 661. The first segment 661 is formed so as to extend along and on the longitudinal center line A-A and the second segment 662 has a generally elliptical shape partially overlapping the first segment 661 and defined on the side of the front waist region 621 about a transverse center line B-B bisecting a dimension of the receiver 603 in the longitudinal direction Y. Such alignment check mark 606 may be formed, for example, by printing the backsheet 636 with appropriate ink or the other coating material.

Now the manner in which the aforementioned arrangement operates in response to occurrence of urination will be described. Upon occurrence of urination, the urine discharged infiltrates into the topsheet 532. The barrier cuffs 507, 507 formed along the side edges 533, 533 of the topsheet prevent the urine from leaking outward in the transverse direction X.

The urine having infiltrated into the topsheet 632 quickly come in contact with the moisture detecting sensors 641 through the spacer 642 and the filter 643. When urine comes in contact with the paired moisture detecting sensors 641 so as to connect these sensors with each other, these moisture detecting sensors 641 are turned on and transmit signals to the control unit 600C. In response to this signal, the control unit 600C actuates the suction pump 600P. Upon actuation of the suction pump 600P, the space 654 of the urine receptacle 605 is subjected to a suction effect via a suction unit 655.

The urine having reached the moisture detecting sensor 641 infiltrates into the dispersant sheet 649 through the cushion sheet 644. The urine having dispersant sheet 649 over a wide range simultaneously infiltrates into the air permeable-resistant sheet 650 which covers the opening of the urine receptacle 605 to define the space 654 therein. When the space 654 is subjected to the suction effect by the suction pump 600P and the opening is covered with the air permeable-resistant sheet 650 wetted with urine, a negative pressure is generated within the space 654. As a result, the urine having infiltrated in the air permeable-resistant sheet 650 is sucked into the space 654 and the urine sucked into the space 654 is sucked through the suction tube 655 from the urine receptacle 605. In this way, immediately after occurrence of urination, the discharged urine can be quickly evacuated out of the wearing article.

In the aforementioned wearing article, the receiver 603, eventually, the alignment check mark 606 formed on the receiver 603 can be visually recognized through the chassis 602. Therefore, it is possible for the care personnel to determine whether the receiver 603 is displaced or not on the basis of the position of the alignment check mark 606 in the course of putting the wearing article 601 on the wearer's body. Occurrence of urination is detected by the moisture detecting sensor 641 when urine comes in contact with a pair of the electrodes 645, 645 so as to bridge these paired electrodes 645, 645. In other words, the receiver 603 may be aligned with the intermediate position between the paired electrodes 645, 645 to achieve the highest detection accuracy.

If the receiver 603 is displaced toward right or left side of the wearer, there is a possibility that the wearer's urine might be discharged outside the paired electrodes 645, 645. In this case, the paired electrodes 645, 645 are bridged by urine only when the urine sufficiently increases to flow beyond one of the electrodes 645 to the other electrode 645. Consequently, it is impossible for a small quantity of urine to bridge the paired electrodes 645, 645 and to be detected. In contrast, if the wearer's urine is discharged just between the paired electrodes 645, 645, it will be easy even for a small quantity of urine to bridge the paired electrodes 645, 645 and to turn them on so that such a small quantity of urine can be reliably detected. In this way, the highest detection accuracy is assured.

According to the present embodiment, the electrodes 645, 645 are formed on both sides of the longitudinal center line A-A and the alignment check mark 606 is formed along the longitudinal center line A-A. With such arrangement, the highest urine detection accuracy can be assured by putting the article on the wearer's body so that the mark 606 may be aligned with the wearer's urethral region.

The alignment check mark 606 comprises the first segment 661 and the second segment 662 so that the receiver 603 may be aligned with the wearer's urethral region particularly by using the second target as a guide. More specifically, the care personnel may place his or her finger on the second segment 662 and press the finger against the wearer's urethral region through the receiver 603 to confirm the wearer's urethral region to align the second segment 662 of the mark 606 with the wearer's urethral region. The alignment check mark 606 is positioned between the paired electrodes 645, 645 and assures it to position the wearer's urethral region in alignment with the position just between the paired electrodes 645, 645.

The first segment 661 of the mark 606 extends in the longitudinal direction Y and makes it possible to determine whether the receiver 603 is displaced or not over a wide range in the longitudinal direction Y. Considering that the urethral region is generally located along the longitudinal center line of the wearer's body, for example, the first segment may be aligned with the navel in the front waist region 621 and may be aligned with the bottom cleavage in the rear waist region 622 to align the intermediate region between the electrodes 645, 645 generally with the longitudinal center line of the wearer's body and thereby with the wearer's urethral region.

As has previously been described, it is possible after the article has been put on the wearer's body to determine the relative position between the longitudinal center line A-A of the receiver 603 and the wearer's body. Therefore, it is possible to correct the displacement of the receiver 603, if it has been determined. In this way, it is possible to avoid the problem that the moisture detecting sensor 641 of the moisture detecting structure 604 might be out of alignment with the wearer's urethral region and discharged urine could not be detected.

While the wearer's article is provided with the moisture detecting sensor 641 serving for detection of urine according to the present embodiment, it is possible to incorporate, in addition to the moisture detecting sensor 641, a feces detecting sensor. While the mesh pants are used as the chassis 602 according to the present embodiment, it is also possible to use widely used means such as disposable diapers or diaper covers as the chassis 602.

While the alignment check mark 606 is formed only on the receiver 603 according to the present embodiment, it is possible to form the mark 606 on the chassis 602 also. In this case, the chassis 602 may be formed with the mark 606 extending in the longitudinal direction Y along the longitudinal center line of the chassis 602 and thereby the position of the chassis 602 can be adjusted so that the mark on the chassis 602 may be aligned with the mark on the receiver 603. Generally, the chassis 602 is scarcely displaced and the receiver 602 may be positioned in alignment with the mark of the chassis 602 to assure that the receiver 603 is positioned on the center line of the wearer's body.

While the second segment 662 of the mark 606 is positioned on the side of the front waist region 621 relatively to the transverse center line B-B according to the present embodiment, it is not essential. For example, the position of the second segment 662 may be appropriately changed depending on various factors such as the size of the wearing article or the receiver 603 or gender of the wearer. It should be appreciated that the second segment is not essential to be formed and, in contrast, two or more second segments may be formed, if desired.

### Second Embodiment

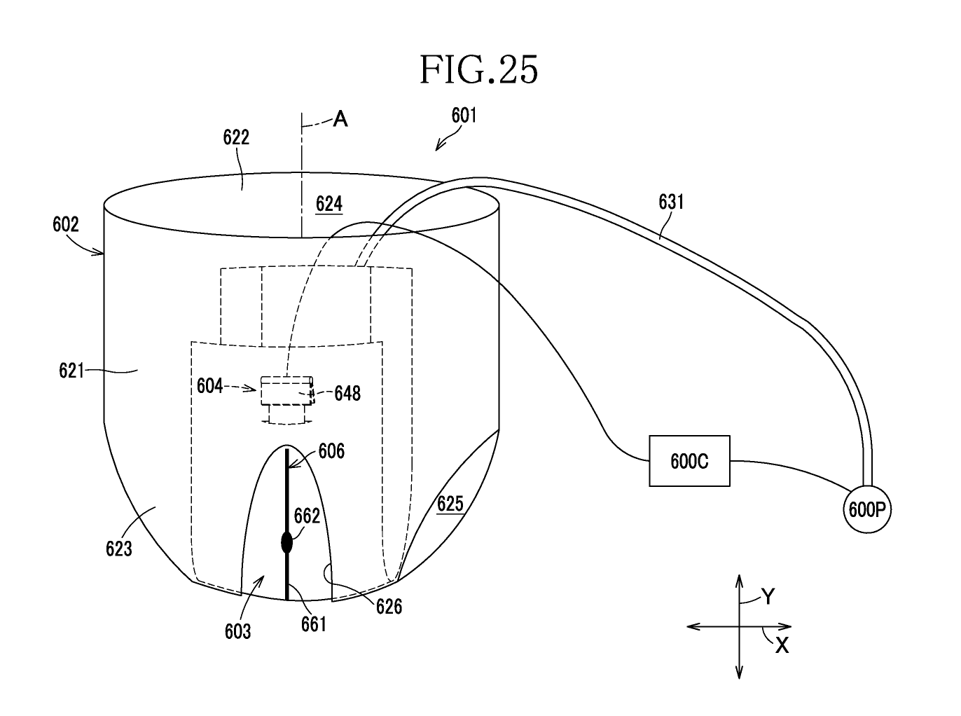

FIG. 25 is a diagram illustrating a second embodiment of the present invention. As will be seen in FIG. 25, the chassis 602 is formed in the crotch region 623 with a window 626 in which the receiver 603 is exposed. The window 626 allows the alignment check mark 606 formed on the receiver 603 to be visually recognized even if the chassis 602 is made of material having no see-through properties.

It is also possible to form the chassis 602 as a whole by material having no see-through properties and to form the crotch region 623 only by materials having see-through properties. For example, the chassis 602 may be formed of an opaque nonwoven fabric made of thermoplastic synthetic resin and a desired region of this nonwoven fabric may be heated under pressure to make it filmy and see-through. Alternatively, the chassis 602 may be formed of an opaque film made of thermoplastic synthetic resin containing inorganic fillers and a desired region of this film may be heated under pressure to smooth such region, to reduce optical diffuse reflection and thereby to make the region see-through. In this case, this film may be laminated together with the nonwoven fabric to reinforce this film or to provide the outer surface with a fabric-like touch.

Whatever the case may be, displacement of the receiver 603 relative to the wearer's urethral region can be checked and thereby it is assured that occurrence of urination can be detected.

1. A urine receiver comprising:
a leakage-barrier receptacle having a bottom and a peripheral wall and a top opening;
a urine evacuation duct extending through said peripheral wall and adapted to be connected with a urine suction means provided separately of said leakage-barrier receptacle;
a plurality of liquid-pervious sheets stacked one on another to cover said top opening; and
a pair of electrodes spaced from each other and extending in one direction in parallel to each other wherein, in response to electrical connection of said pair of electrodes via urine contained in one or more of said plurality of liquid-pervious sheets, said urine suction means is actuated to suck the urine discharged by a wearer into said leakage-barrier receptacle through said top opening and to evacuate said urine sucked into said leakage-barrier receptacle from said leakage-barrier receptacle through said urine evacuation duct, wherein,
any one of said plurality of liquid-pervious sheets lying outside said pair of electrodes in a depth direction of said leakage-barrier receptacle is formed of a nonwoven fabric containing thermoplastic synthetic fibers and has an outer surface facing outward in said depth direction and an inner surface facing inward in said depth direction wherein said outer surface is formed with undulations comprising crests and troughs alternating in said one direction and each of said crests and troughs extends in a direction intersecting with said one direction across said pair of electrodes.

2. A wearing article having a longitudinal direction and a transverse direction and comprising:
a side facing a wearer's body;
a side facing a wearer' garment;
a chassis having a front waist region, a rear waist region and a crotch region extending between said front and rear waist regions; and
a receiver adapted to receive bodily fluids discharged onto said chassis wherein the bodily fluids received by said receiver are sucked by a moisture sucking device out of the wearing article,
said receiver comprises a receptacle connected to said moisture suction device for collecting bodily fluids received by said receiver, and a moisture-absorbent structure lying on a side of said receptacle facing the wearer's garment; and
said moisture-absorbent structure comprises a moisture-pervious sheet lying on a side of said receptacle facing the wearer's body, a moisture-impervious sheet lying on the said side facing the wearer's garment and a moisture-absorbent material sandwiched between said moisture-pervious sheet and said liquid-impervious sheet.

3. A wearing article having a longitudinal direction and a transverse direction and comprising:
a side facing a wearer's body;
a side facing a wearer's garment;
a chassis having a front waist region, a rear waist region and a crotch region extending between said front and rear waist regions; and
a urine receiver adapted to receive bodily fluids discharged onto said chassis wherein bodily fluids received by said urine receiver are sucked by a moisture sucking device out of the wearing article,
said receiver is formed with visible alignment check mark adapted to be visually recognized from the outside through said chassis.
